SEQUENCE LISTING (18 sequences)

SEQ ID NO: 1 - protein, 458 aa, Homo sapiens
MRGPAVLLTVALATLLAPGAGAPVQSQGSQNKLLLVSFDGFRWNYDQDVDTPNLDAMARDGVKARYMTPAFVTMTSPCHFTLVTGKYIENHGVVHNMYYNTTSKVKLPYHATLGIQRWWDNGSVPIWITAQRQGLRAGSFFYPGGNVTYQGVAVTRSRKEGIAHNYKNETEWRANIDTVMAWFTEEDLDLVTLYFGEPDSTGHRYGPESPERREMVRQVDRTVGYLRESIARNHLTDRLNLIITSDHGMTTVDKRAGDLVEFHKFPNFTFRDIEFELLDYGPNGMLLPKEGRLEKVYDALKDAHPKLHVYKKEAFPEAFHYANNPRVTPLLMYSDLGYVIHGRINVQFNNGEHGFDNKDMDMKTIFRAVGPSFRAGLEVEPFESVHVYELMCRLLGIVPEANDGHLATLLPMLHTESALPPDALLVADGPCLPSLSQAKGCMPLSPAAPTPAWLLWCW

SEQ ID NO: 2 - DNA, 1701 bp, Homo sapiens
gtccatctggaaggcccagcatgagaggcccggccgtcctcctcactgtggctctggccacgctcctggctcccggggccggagcaccggtacaaagtcagggctcccagaacaagctgctcctggtgtccttcgacggcttccgctggaactacgaccaggacgtggacacccccaacctggacgccatggcccgagacggggtgaaggcacgctacatgacccccgcctttgtcaccatgaccagcccctgccacttcaccctggtcaccggcaaatatatcgagaaccacggggtggttcacaacatgtactacaacaccaccagcaaggtgaagctgccctaccacgccacgctgggcatccagaggtggtgggacaacggcagcgtgcccatctggatcacagcccagaggcagggcctgagggctggctccttcttctacccgggcgggaacgtcacctaccaaggggtggctgtgacgcggagccggaaagaaggcatcgcacacaactacaaaaatgagacggagtggagagcgaacatcgacacagtgatggcgtggttcacagaggaggacctggatctggtcacactctacttcggggagccggactccacgggccacaggtacggccccgagtccccggagaggagggagatggtgcggcaggtggaccggaccgtgggctacctccgggagagcatcgcgcgcaaccacctcacagaccgcctcaacctgatcatcacatccgaccacggcatgacgaccgtggacaaacgggctggcgacctggttgaattccacaagttccccaacttcaccttccgggacatcgagtttgagctcctggactacggaccaaacgggatgctgctccctaaagaagggaggctggagaaggtgtacgatgccctcaaggacgcccaccccaagctccacgtctacaagaaggaggcgttccccgaggccttccactacgccaacaaccccagggtcacacccctgctgatgtacagcgaccttggctacgtcatccatgggagaattaacgtccagttcaacaatggggagcacggctttgacaacaaggacatggacatgaagaccatcttccgcgctgtgggccctagcttcagggcgggcctggaggtggagccctttgagagcgtccacgtgtacgagctcatgtgccggctgctgggcatcgtgcccgaggccaacgatgggcacctagctactctgctgcccatgctgcacacagaatctgctcttccgcctgatgctctgctggtcgcggacggaccctgcctccccagcttatcccaggccagaggctgcatgccactgtccccggcagcgccaacccctgcttggctgttatggtgctggtaataagcctgcagcccaggtccaaagcccccggcgagccggtcccataaccggccccctgcccctgcccctgctcctgctcctccccttcgggccccctcctcctgcaaaacccgctcccgaagcggcgctgccgtctgcagccacgcgggggcgcgcgggagtcttctgcgggcgctggaacctgcagacccggcctcggtcagctgggaggggcccgccccggcacaaagcacccatgggaataaaggccaagccgcgacagtcagcaaaaaaaaaaaaaaaaaaaaaaaaaaaaaaaaaaaaa

SEQ ID NO: 3 - protein, 18 aa, Homo sapiens
AFVTMTSPCHFTLVTGKY

SEQ ID NO: 4 - protein, 458 aa, Homo sapiens
MRGPAVLLTVALATLLAPGAGAPVQSQGSQNKLLLVSFDGFRWNYDQDVDTPNLDAMARDGVKARYMTPAFVTMTSPCHFTLVTGKYIENHGVVHNMYYNTTSKVKLPYHATLGIQRWWDNGSVPIWITAQRQGLRAGSFFYPGGNVTYQGVAVTRSRKEGIAHNYKNETEWRANIDTVMAWFTEEDLDLVTLYFGEPDSTGHRYGPESPERREMVRQVDRTVGYLRESIARNHLTDRLNLIITSDHGMTTVDKRAGDLVEFHKFPNFTFRDIEFELLDYGPNGMLLPKEGRLEKVYDALKDAHPKLHVYKKEAFPEAFHYANNPRVTPLLMYSDLGYVIHGRINVQFNNGEHGFDNKDMDMKTIFRAVGPSFRAGLEVEPFESVHVYELMCRLLGIVPEANDGHLATLLPMLHTESALPPDGRPTLLPKGRSALPPSSRPLLVMGLLGTVILLSEVA

SEQ ID NO: 5 - DNA, 1878 bp, Homo sapiens
  misc_feature (905)..(905): n is a, c, g, or t
gtccatctggaaggcccagcatgagaggcccggccgtcctcctcactgtggctctggccacgctcctggctcccggggccggagcaccggtacaaagtcagggctcccagaacaagctgctcctggtgtccttcgacggcttccgctggaactacgaccaggacgtggacacccccaacctggacgccatggcccgagacggggtgaaggcacgctacatgacccccgcctttgtcaccatgaccagcccctgccacttcaccctggtcaccggcaaatatatcgagaaccacggggtggttcacaacatgtactacaacaccaccagcaaggtgaagctgccctaccacgccacgctgggcatccagaggtggtgggacaacggcagcgtgcccatctggatcacagcccagaggcagggcctgagggctggctccttcttctacccgggcgggaacgtcacctaccaaggggtggctgtgacgcggagccggaaagaaggcatcgcacacaactacaaaaatgagacggagtggagagcgaacatcgacacagtgatggcgtggttcacagaggaggacctggatctggtcacactctacttcggggagccggactccacgggccacaggtacggccccgagtccccggagaggagggagatggtgcggcaggtggaccggaccgtgggctacctccgggagagcatcgcgcgcaaccacctcacagaccgcctcaacctgatcatcacatccgaccacggcatgacgaccgtggacaaacgggctggcgacctggttgaattccacaagttccccaacttcaccttccgggacatcgagtttgagctcctggactacggaccaaacgggatgctgctccctaaagaagggaggctggagaangtgtacgatgccctcaaggacgcccaccccaagctccacgtctacaagaaggaggcgttccccgaggccttccactacgccaacaaccccagggtcacacccctgctgatgtacagcgaccttggctacgtcatccatgggagaattaacgtccagttcaacaatggggagcacggctttgacaacaaggacatggacatgaagaccatcttccgcgctgtgggccctagcttcagggcgggcctggaggtggagccctttgagagcgtccacgtgtacgagctcatgtgccggctgctgggcatcgtgcccgaggccaacgatgggcacctagctactctgctgcccatgctgcacacagaatctgctcttccgcctgatggaaggcctactctcctgcccaagggaagatctgctctcccgcccagcagcaggcccctcctcgtgatgggactgctggggaccgtgattcttctgtctgaggtcgcataacgccccatggctcaaggaagccgccgggagctgcccgcaggccctgggccggctgtctcgctgcgatgctctgctggtcgcggacggaccctgcctccccagcttatcccaggccagaggctgcatgccactgtccccggcagcgccaacccctgcttggctgttatggtgctggtaataagcctcgcagcccaggtccagagcccccggcgagccggtcccataaccggccccctgcccctgcccctgctcctgctcctccccttcgggccccctcctcctgcaaaacccgctcccgaagcggcgctgccgtctgcagccacgcgggggcgcgcgggagctctgcgggcgctggaacctgcagacccggcctcggtcagctgggaggggcccgccccggcacaaagcacccatgggaataaaggccaagccgcgacagtcagcaaaaaaaaaaaaaaaaaaaaaaaaaaaaaaaaaaaaa

SEQ ID NO: 6 - protein, 415 aa, Homo sapiens
MRGPAVLLTVALATLLAPGAGAPVQSQGSQNKLLLVSFDGFRWNYDQDVDTPNLDAMARDGVKARYMTPAFVTMTSPCHFTLVTGKYIENHGVVHNMYYNTTSKVKLPYHATLGIQRWWDNGSVPIWITAQRQGLRAGSFFYPGGNVTYQGVAVTRSRKEGIAHNYKNETEWRANIDTVMAWFTEEDLDLVTLYFGEPDSTGHRYGPESPERREMVRQVDRTVGYLRESIARNHLTDRLNLIITSDHGMTTVDKRAGDLVEFHKFPNFTFRDIEFELLDYGPNGMLLPKEGRLEKVYDALKDAHPKLHVYKKEAFPEAFHYANNPRVTPLLMYSDLGYVIHGRINVQFNNGEHGFDNKDMDMKTIFRAVGPSFRAGLEVEPFESVHVYELMCRLLGIVPEANDGHLATLLPMLHT

SEQ ID NO: 7 - protein, 10 aa, Homo sapiens
FVTMTSPCHF

SEQ ID NO: 8 - protein, 8 aa, Homo sapiens
FVTMTSPC

SEQ ID NO: 9 - protein, 7 aa, Homo sapiens
PTKTFPN

SEQ ID NO: 10 - DNA, 27 bp, Artificial
  chemically synthesized expression tag
ggcccagcatgagaggcccggccgtcc

SEQ ID NO: 11 - DNA, 27 bp, Artificial
  chemically synthesized expression tag
ggacggccgggcctctcatgctgggcc

SEQ ID NO: 12 - DNA, 20 bp, Artificial
  chemically synthesized primer
taatacgactcactataggg

SEQ ID NO: 13 - DNA, 18 bp, Artificial
  chemically synthesized primer
tccgagatctggacgagc

SEQ ID NO: 14 - DNA, 40 bp, Artificial
  chemically synthesized primer
ggcccgagacggggtgaaggcacgctacatgacccccgcc

SEQ ID NO: 15 - DNA, 23 bp, Artificial
  chemically synthesized primer
tggcccgtggagtccggctcccc

SEQ ID NO: 16 - protein, 15 aa, Homo sapiens
KLLLVSFDGFRWNYD

SEQ ID NO: 17 - DNA, 31 bp, Artificial
  chemically synthesized primer
atggatccatgagaggcccggccgtcctcct

SEQ ID NO: 18 - DNA, 31 bp, Artificial
  chemically synthesized primer
acgtcgacttaccagcaccataacagccaag

(19) United States
(12) Patent Application Publication
(10) Pub. No.: US 2007/0092503 A1
(43) Pub. Date: Apr. 26, 2007

(54) Human alkaline sphingomyelinase and use thereof

(76) Inventors: Tomas Bergman, Jarfalla (SE); Rui-Dong Duan, Lund (SE); Ake Nilsson, Lund (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 10/553,906

(22) PCT Filed: Apr. 23, 2004
(86) PCT No.: PCT/SE04/00628

§ 371 Date: Oct. 21, 2005

Related U.S. Application Data
(60) Provisional application No. 60/320,139, filed on Apr. 24, 2003.
(60) Provisional application No. 60/481,598, filed on Nov. 5, 2003.

(51) Int. Cl. A61K 38/46; C12Q 1/68; C07H 21/04; C12P 21/06; C12N 9/20

(52) U.S. Cl. 424/94.6; 435/6; 435/69.1; 435/198; 435/320.1; 435/325; 536/23.2

(57) ABSTRACT

An isolated human alkaline sphingomyelinase (Alk-Smase) or a variant thereof is capable of hydrolysing sphingomyelin. Methods are provided for isolating human Alk-Smase and for preparing human recombinant Alk-Smase. Further methods are provided for the use of the enzyme for the treatment of colon cancer.

### TECHNICAL FIELD

This invention relates to a novel human alkaline sphingomyelinase (Alk-Smase) capable of hydrolysing sphingomyelin at an alkaline pH. The invention also relates to a composition comprising the protein and methods for preparing the protein.

### BACKGROUND OF THE INVENTION

Ceramides and other shingolipid metabolites such as sphingosine and sphingosine-1-phosphate signalling substances are involved in a variety of cellular responses, including cell differentiation, cell cycle suspension, cell ageing, apoptosis, etc. Ceramides are produced mainly as a result of a hydrolysis of sphingomyelin, a membrane sphingophospholipid located mainly in the plasma membrane and in the lysosome membrane and the brush borders of epithelial cells.

Smase hydrolyses the phosphodiester bond of sphingomyelin to generate ceramide and phosphocholine. Different Smases have been described in eukaryotes and prokaryotes and are distinguished by their localization, pH optima, and requirement for metal ions. However, only a few enzymes have been characterized at molecular level. The best characterized of these enzymes is the acidic Smase and the bacterial Mg2+ dependent neutral Smase (T Levade and J P Jaffrezou Biochim Biophys Acta 1999; 1438:1-17, Y Matsou et al; Protein Sci 1996; 5:2459-2467). Purification of mammalian neutral Smases involved in cell signalling has proved very difficult (F Rodrigues-Lima et al J Biol Chem 2000; 275:28316-325). A putative clone was isolated but the expression resulted in only a modest increase in hydrolysis of exogenous sphingomyelin (S Chatterjee et al J Biol Chem 1999; 274:37407-37412) Other mammalian clones have been isolated on the basis of their sequence similarity with bacterial neutral Smase (Tomiuk et al Proc Natl Acad Sci 1998; 95:3638-3643). The structural requirements for catalysis and membrane targeting of mammalian enzymes with neutral Smase and lysophospholipase C activities have been characterized, and exhibit no similarity with the structure necessary for the action of Alk-Smase disclosed herein (F Rodrigues-Lima et al J Biol Chem 2000; 275:28316-325). Various Smases have been identified, e.g. lysosomal acid Smase with acidic pH-optimum (A-Smase), cytoplasmic Zn-dependent A-Smase (S L Schissel et al J Biol Chem 1998; 273:18250-259), alkaline pH-optimum Smase (Alk-Smase)(A Nilsson, R D Duan Chem Phys Lipids 1999; 102:97-105), cytoplasmic Mg²⁺-dependent neutral pH-optimum Smase (N-cSmase), and membrane associated Mg²⁺-dependent neutral pH-optimum Smase (N-mSmase) (T Levade and J P Jaffrezou Biochim Biophys Acta 1999; 1438:1-17)(S Chattedee; Chem Phys Lipids 1999; 102:79-96) 1999).

Due to the problem of purifying and sequencing Smases involved in cell signalling (F Rodrigues-Lima J Biol Chem 2000; 275:28316-325), some of the Smases are only characterised and described based on their inherent activity at different pH optimum, not on their chemical and structural properties based on DNA and/or peptide information, knowledge of active site, structural information, etc.

The presence of a Smase activity in the gut with alkaline pH optimum has been designated alkaline Smase. A ceramidase that catalysed the further degradation of ceramide to sphingosine and free fatty acid was first described in 1969 (Å Nilsson, Biochim. Biophys. Acta 1969; 176:339-47). Pancreatic enzymes that efficiently hydrolysed sphingolipids were found to be lacking. Pancreatic bile salt stimulated lipase was shown to have some ceramidase activity (L Nyberg et al J Pediatr Gastroenterol Nutr 1998; 27:560-567) In vivo studies further showed that dietary sphingomyelin was sequentially degraded, first to ceramide and phosphocholine; the amide bond of ceramide was then hydrolysed to sphingosine and free fatty acids. The sphingosine formed was efficiently absorbed and oxidized to palmitic acid in the intestinal mucosa. One portion is reacylated into ceramide and more complex sphingolipids (Å Nilsson Biochim. Biophys Acta 1968:164:575-84 and E Schmelz et al J Nutr 124:702-712 and L Nyberg et al J Nutr Biochem 1997; 8:112-1118). Glucosylceramide and galactosylceramide were shown to be digested and absorbed in a similar way (Å Nilsson Biochim Biophys Acta 1969; 187:113-121). Alk-Smase and intestinal ceramidase have then been studied regarding enzymatic and biochemical properties, and physiological role. Both Alk-Smase and ceramidase are enriched in the brush border but also released into the gut lumen (Å Nilsson, Biochim. Biophys. Acta 1969; 176:339-47). The hydrolysis of respective substrates are strongly bile salt dependent Y Cheng et al J Lipid Res 2002; 43:316-24). Alk-Smase is extremely resistant to pancreatic proteases and significant amounts of Alk-Smase and intestinal ceramidase are found in small intestinal contents (R D Duan et al J Lipid Res 2003; and R D Duan et al Lipids 2001; 36:807-12). The activity of Alk-Smase is low in duodenum, highest in the middle and lower small intestine and lower but distinctly expressed in the colon (Duan et al Dig Dis Sci 1996; 41:1801-6). Most sphingomyelin digestion occurs in the lower and the middle of the small intestine. The digestion is incomplete and extended throughout the whole length of the small intestine. The colon is exposed to increased amounts of unhydrolyzed sphingomyelin and ceramide when dietary sphingomyelin is ingested. Due to its pronounced resistance to pancreatic proteases, Alk-Smase (R D Duan, A Nilsson Methods Enzymol 2000; 311-276-86) is not degraded in the small intestinal content. This is demonstrated by the finding that levels found in the intestinal content collected from ileostomy patients are so high that the ileostomy content has been successfully used for purifying Alk-Smase (R D Duan et al J Lipid Res 2003). Thus, colon mucosa contains Alk-Smase, and is also exposed to Alk-Smase passing from the small intestine into the colon.

Thus, Alk-Smase acts throughout the small intestine and colon to generate ceramide from exogenous and endogenous sphingomyelin. The ceramide may be further degraded by the intestinal mucosal ceramidase found by us. As a result ceramide, sphingosine and sphingosine-1-phosphate levels may be affected by the amount of Alk-Smase and ceramidase and by the amount of substrates available in the diet.

Development of colon cancer and inflammation in the gut involves a complex interaction between genetic and environmental factors. Inflammatory bowel diseases, i.e., Crohns disease, ulcerative colitis and microscopic colitis, are common diseases caused by a genetic predisposition that enhances the inflammatory response to normal colonic bacteria. A number of signalling systems are involved among which are several cytokines and lipid messengers. In colon cancer, COX2 catalyzing prostaglandin formation is often increased and leukotriene D4 receptors are induced, this leukotriene being found to be an antiapoptotic factor. A supply of sphingomyelin or glycosphingolipids in their diet counteracts development of colon cancer in mice treated with the chemical carcinogen dimethylhydrazine (D L Dillehay et al J Nutr 1994; 124:615-20. and E M Schmelz et al Cancer Res 1996; 56:4936-41 and Nutr Cancer 1997; 28:81-5 and Cancer Res 1999; 59:5768-72 and J Nutr 2000; 130:522-7). Sphingoid bases were found to influence growth and apoptosis in colon cells by signalling systems known to be important in development of colon cancer (E M Schmelz et al Cancer Res 2001; 61:6723-9.

Alk-Smase activity is lowered in colon tumours (E Hertervig et al Cancer 1997; 79:448-53) and in familial adenomatous polyposis (E Hertervig et al Br J Cancer 1999; 81:232-6). The success of continued work exploring the possibilities to influence tumour development and inflammation depend on knowledge of the specific structure and gene expression of the enzyme, which is currently unknown. Knowledge of the specific structure and gene expression may also be the basis for production of bacterial enzymes having properties analogous to human Alk-Smase.

Characterization of human Alk-Smase activity has involved the following steps and publications:

The longitudinal distribution shows highest activity levels in jejunum and ileum but the enzyme occurs also in the colon (R D Duan et al Biochim. Biophys. Acta 1995; 1259:49-55 and R D Duan et al Dig. Dis. Sci 1996; 41:1801-6).

The enzyme has been purified from rat small intestine and characterized enzymologically (Y Cheng et al, J Lipid Res 2002; 43:316-24). Alk-Smase has been partially purified from an eluate obtained by luminal elution with saline containing bile salts and the obtained eluate has been used as a starting material enriched in Alk-Smase.

The human intestinal Alk-Smase has been purified and expression in colon tumours and adjacent mucosa has been studied by measuring enzyme activity and immunoreactive mass of enzyme protein (R D Duan et al J Lipid Res 2003; 278:38528-36). Alk-Smase has been purified from human ileostomy content which is possible due to its extreme resistance to pancreatic proteolytic enzymes. Using bile salt eluate in the rat (Y Cheng et al J Lipid Res 2002; 43:316-24) and ileostomy content in humans the difficulties of purifying the enzyme from homogenates of intestinal mucosa can be avoided. The latter approach did not succeed due to the presence of proteins with similar chromatographic behaviour (R D Duan et al J Lipid Res 2003; 278:38528-36).

The enzyme occurs in human bile and has been partially purified there from. (L Nyberg et al Biochim.Biophys.Acta 1996; 1300:42-8. RD Duan, Å Nilsson Hepatology 1997; 26:823-30). Obtaining the sequence from the bile enzyme has met with difficulties due to the limited amounts of enzyme that can be obtained and the difficulties in removing contaminating proteins.

The enzyme activity level is lower in colon tumours than in surrounding mucosa (E Hertervig et al Cancer 1997; 79:448-53).

The enzyme activity level is low in patients with familial colon polyposis (E Hertervig et al Br J Cancer 1999; 81:232-6).

There exists an intestinal ceramidase with specific properties and activity, although milk bile salt stimulated lipase—in addition to its action on several glycerides—has some ceramidase activity as well (P Lundgren et al Dig Dis Sci 2001; 46:316-24. RD Duan et al Lipids 2001; 36:807-12). Clearly the intestinal ceramidase differs from the bile salt stimulated lipase and catalysis most in the ceramide digestion.

The success of continued work exploring the possibilities to influence, e.g., tumour development and inflammation, depends on the knowledge of the specific structure and gene expression of the enzyme. This knowledge may also be the basis for large scale production of the enzyme in bacteria.

Thus, it is highly desirable in the light of aforementioned problems to develop means and methods for isolation and large scale preparation of Alk-Smases, to be able to gain more knowledge and characterise the enzyme so as to enable development of means and methods for treatment of Smase-related deficiencies/diseases, such as celiac disease where the Alk-Smase activity is low due to the villous atrophy, in ulcerative colitis where the cancer risk is increased during long term follow up and in colon cancer, in the irritable bowel syndrome, and in patients running an increased risk of hereditary forms of colon cancers. Preterm infants are vulnerable to necrotizing enteritis. The risk is reduced through consumption of by human milk since sphingomyelin is a major phospholipid in milk. Cancers in the breast, prostate, lungs, skin, liver, stomach, thyroid gland, small bowel, pancreas and malignant tumours in lymphoid tissues, the musculo-skeletal system and brain are also of interest. The present invention addresses these needs and interests.

### SUMMARY OF THE INVENTION

In view of the foregoing disadvantages known in the art when trying to isolate and characterise Alk-Smases for developing means and methods for treatment of diseases related to Smase deficiencies or where Smase may exert beneficial effects such as celiac disease, Crohns disease, ulcerative colitis, irritable bowel syndrome, in aforementioned types of tumours, and neonatal immaturity in the gut, the present invention provides purified human alkaline. Despite difficulties in isolating Alk-Smase, the present inventors have succeeded and fully characterised human Alk-Smase. Human Alk-Smase's DNA and corresponding amino acid sequence has been identified and isolated as well as characterised due to its function.

An object of the present invention is thus to provide a protein comprising the sequence Seq ID No 1 or Seq ID No 4, or a variant or part thereof, capable of hydrolysing Smase.

Said protein or variant thereof is capable of hydrolysing sphingomyelin at a pH of 7.5-9.

Said protein or variant thereof may further have >50% of its hydrolysing activity at a pH>7.5.

The present invention also provides a nucleotide sequence encoding the protein mentioned above.

Said nucleotide sequence may comprise the sequence Seq ID No 2 or Seq ID No 5, or a variant or part thereof.

Furthermore, the present invention provides a recombinant expression and secretion vector comprising a polynucleotide encoding a secretion signal peptide; a DNA sequence which promotes transcription in a host cell located upstream from the polynucleotide encoding the secretion signal peptide; a DNA sequence encoding a protein according to the invention in a translation reading frame with said polynucleotide encoding the secretion signal peptide; and a transcription terminator sequence located downstream from the DNA sequence encoding said protein.

Still furthermore, the present invention provides a host cell comprising said recombinant expression system from which Alk-Smase is expressed.

Said host cell may be a bacteria, a mammalian cell or a yeast cell which in the absence of said recombinant expression system, does not normally produce an Alk-Smase.

Still furthermore, the present invention provides a method for isolation of human Alk-Smase protein. The method comprises the steps of
i) providing a small intestinal or colon content from a human,
ii) homogenising the small intestinal or colon content,
iii) purifying Alk-Smase using DEAE Sephadex chromatography,
iv) purifying using Uno anion exchange chromatography,
v) purifying using hydrophobic chromatography,
   thereby isolating the human Alk-Smase protein.

Still furthermore, a method for preparation of human recombinant Alk-Smase protein capable of hydrolysing sphingomyelin. Said method comprises the steps of
i) providing a host cell and a host cell growth medium,
ii) preparing a host cell culture;
iii) culturing the host cell culture and
iv) harvesting the host cell culture and recovering the human recombinant Alk-Smase.

Said method may recover human Alk-Smase protein either from the culture medium, the host cells or after separating the host cells from the culture medium.

Still furthermore, an isolated human Alk-Smase protein, comprising the protein described herein having an active site with amino acid sequence AFVTMTSPCHFTLVTGKY (Seq ID No 3), particularly FVTMTSPCHF (Seq ID No 7), or a variant thereof is disclosed.

Furthermore, the present invention also provides a composition comprising a protein according to the invention, or a nucleic acid according to the invention, or a human isolated Alk-Smase according to the invention, and a biocompatible carrier or additive.

Furthermore, uses of said protein or nucleic acids according to the invention are included for the preparation of a pharmaceutical composition for the treatment of colon cancer.

Furthermore, a kit comprising the protein according to the invention or the isolated protein according to the invention, and a stabiliser is included.

The disclosed information may be used to clone the enzyme and there are sequence homologies that also make it possible to clone rat and mouse alkaline Smase based on the knowledge provided herein.

This knowledge will further make it possible to prepare gene knockout mice, production of large amounts of recombinant enzyme and diagnosis of the genetic polymorphism in humans.

### SHORT DESCRIPTION OF DRAWINGS

FIG. 1 shows the purity of human and rat intestinal Alk-Smase. The enzyme was purified by DEAE-anion exchange chromatography, Phenyl Sepharose hydrophobic interaction chromatography, Uno Q high affinity anion chromatography, native electrofocusing, and gel chromatography. Lane A: standard proteins, lane B: original material for purification of human Alk-Smase, lane C: purified human intestinal Alk-Smase, and lane D: purified rat intestinal Alk-Smase.

FIG. 2 shows the 458 amino acid sequence of human Alk-Smase (Seq ID No 1).

FIGS. 3a and 3b show the nucleotide sequence of human Alk-Smase cDNA (Seq ID No 2). The sequence from 10 to 30 is a part that is not translated. The sequence before nt 10 originates from the vector and is not shown. The sequence from 31 to 1404 is the reading frame which encodes a 458 amino acid (Seq ID No. 1). The sequence from 1407 to 1676 is not translated. The sequence after 1676 is a poly A tail.

FIGS. 4a-c show the characteristics of human Alk-Smase. FIG. 4a shows the optimal pH of the enzyme. The activity was low at pH less than 6 and sharply increased when pH is 7 or higher. The maximal activity was obtained at pH 8.5, the activity at pH 7.5 being about 68% of the maximal activity. FIG. 4b shows that the activity at alkaline pH without divalent cations was significantly higher than at 7.5 with Mg present. FIG. 4c shows that relatively high activities were detected under Ca2⁺ and Mg2⁺ free conditions. FIG. 4d shows that Zn2⁺, which can activate other types of Smase in serum and in the arterial wall, significantly inhibited Alk-Smase activity with a 50% inhibition at 0.015 mM.

FIG. 5 shows the effect of bile salt on human Alk-Smase. The activities were determined in the presence of different concentrations of bile salts. The maximal stimulated effects of each bile salt are shown in the figure. Abbreviations include taurocholate (TC) and taurochenodeoxycholate (TCDC).

FIG. 6 shows that Triton X 100 strongly inhibits human Alk-Smase activity in the presence or absence of TC, right panel. Alk-Smase activity is shown in the presence of different concentrations of Triton X 100 with and without taurocholate (10 mM). Triton X 100 dose dependently inhibits human Alk-Smase.

FIG. 7 shows activity of human Alk-Smase determined in the presence of various concentrations of sphingomyelin (top panel). In the lower panel, the Vmax was determined by Lineweaver-Burk plot. Under these conditions, 1 mg of the enzyme is able to hydrolyze 11 mmole sphingomyelin in one hour.

FIG. 8 shows the cDNA sequence of human Alk-Smase from nucleotide 92-1735. Nucleotide (nt) 1-91 is a part that is not translated or a vector sequence. The sequence after poly A is from the vector.

FIG. 9 shows an alignment of the amino acid sequence of human Alk-Smase with NNP:s, the active region and ion binding site.

FIG. 10 shows the effect of rat Alk-Smase on proliferation of HT29 colon cancer cells. Alk-Smase dose-dependently inhibited cell growth.

FIG. 11 shows a modified amino acid sequence of human intestinal Alk-Smase (Seq ID No 4). Amino acids 1-422 are identical to Seq ID No 1.

FIG. 12 shows a modified nucleotide sequence of human intestinal Alk-Smase (Seq ID No 5). Nucleotides 1-1266 are identical to Seq ID No 2.

FIG. 13 shows levels of Alk-Smase capable of hydrolysing sphingomyelin under optimal conditions for Alk-Smase. Cos 7 cells are transfected with either the wild type of Alk-Smase cDNA (sequence 21-1397 of ID No 5, reading frame) which encodes Seq ID No 4, or transfected with the Alk-Smase cDNA sequence from 21-1285 (C-truncated), which encodes a 415 amino acid sequence (Seq ID No 6). After transfection, the Alk-Smase activity in the medium and in the cell lysate was determined.

### DETAILED DESCRIPTION OF THE INVENTION

Definitions

The term “enzymatic conditions” are intended to mean that any necessary conditions available in an environment, which will permit the enzyme to function.

The term “nucleotide sequence” is intended to mean a sequence of two or more nucleotides. The nucleotides may be of genomic, cDNA, RNA, semi synthetic or synthetic origin or a mixture thereof. The term includes single and double stranded forms of DNA or RNA.

The term “deleted and/or substituted” is intended to mean that one or more amino acid residue(s) is/are removed (deleted) from the polypeptide and/or changed (substituted) into another amino acid(s).

The term “promoter region” is intended to mean one or more nucleotide sequences involved in the expression of a nucleotide sequence, e.g. promoter nucleotide sequences, as well as nucleotide sequences involved in regulation and/or enhancement of the expression of the structural gene. A promoter region comprises a promoter nucleotide sequence involved in the expression of a nucleotide sequence as a protein, and normally other functions such as enhancer elements and/or signal peptides. The promoter region may be selected from a plant, virus and bacteria or it may be of semi-synthetic or synthetic origin or a mixture thereof as long as it functions in a microorganism.

The term “a non-translated region” also called “termination region” is intended to mean a region of nucleotide sequences, which typically cause the termination of transcription and the polyadenylation of the 3′ region of the RNA sequence. The non-translated region may be of native or synthetic origin as long as it functions in a microorganism according to the definition above.

The term “operably linked” is intended to mean the covalent joining of two or more nucleotide sequences by means of enzymatic ligation, in a configuration which enables the normal functions of the sequences ligated to each other. For example a promoter region is operably linked to a signal peptide region and/or a coding nucleotide sequence encoding a polypeptide to direct and/or enable transcription of the coding nucleotide sequence. Another example is a coding nucleotide sequence operably linked to a 3′ non-translated region for termination of transcription of the nucleotide sequence. Generally, “operably linked” means that the nucleotide sequences being linked are continuously and in reading frame. Linking is normally accomplished by ligation at convenient restriction sites. If such sites do not exist, synthetic adaptors or the like are used in conjunction with standard recombinant DNA techniques well known for a person skilled in the art.

The term “alkaline Smase” (Alk-Smase) defines a mammalian sphingomyelin preferring enzyme with a pH optimum of 8.5-9.0, having the capacity to hydrolyse sphingomyeline at a pH 7.4 or above with more than 50% of its maximal hydrolysing capacity remaining. The enzyme is inhibited by several artificial detergents and is not dependent on divalent metal ions in contrast to several neutral and bacterial Smases.

The term “stringent conditions” is intended to mean hybridisation and washing conditions which permits the hybridisation between related nucleotide sequences to be permitted during hybridisation and remain hybridised during the washing.

As used herein, “pharmaceutical composition” means therapeutically effective composition according to the invention.

A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e., a carrier or administration vehicle. Further, it is intended to mean an amount sufficient to reduce, and most preferably to prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent, i.e., carrier or additive. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as are well known in the art.

As used herein, “treating” means treating for curing which may be a full curing or a partial curing of a condition or conditions.

The term “alleviate” is herein intended to mean not only a reduction of intensity of a condition or indication, but also postponing onset of a condition or indication.

The term “prevent” is herein intended to mean to ensure that something does not happen, e.g. that a condition or indication relating to an immature GIT does not happen. By preventing a certain condition or indication, the onset of such condition or indication is postponed.

The expression “comprising” as used herein should be understood to include, but not be limited to, the stated items.

The invention will now be described by way of the following non-limiting examples.

Human Alk-Smase

The present invention provides purified and sequenced human Alk-Smase and the identified gene coding for this protein.

The present invention further concerns the use of defined purified and recombinant mammalian Alk-Smases, more or less modified, in prevention of cancer and inflammatory processes in the gut and liver.

The present invention further involves preparation of recombinant mammalian Alk-Smase.

The present invention further concerns preparation of gene knockout animals.

The present invention further concerns oral, local and intravenous administration of formulations comprising Alk-Smase with and without further additions such as other enzymes including bile salt stimulated intestinal ceramidase (B-cer) or substrates including Sphingomyelin, glycosphingolipids and ceramide with varying fatty acid chain length and with naturally occurring or modified sphingoid bases of varying chain length. The additives may enhance Alk-Smase biological activity thereby increasing the generation of metabolites that may permeate into the cells or be further metabolized.

A protein comprising the sequence Seq ID No 1 or Seq ID No 4, or variants thereof, which variant is capable of hydrolysing sphingomyelin is disclosed.

In one embodiment the protein according to the invention is capable of hydrolysing sphingomyelin at a pH of 7.5-9.

In a further embodiment, the protein according to the invention has >50% of its hydrolysing activity, such as 51, 60, 70, 80, 90, 99, or 100% of its activity, at a pH>7.5.

In one embodiment, the protein according to the invention has >70% of its activity at a pH>8.5.

In still another embodiment, the protein according to the invention has at least 80, 90, 95, 98, 99, 100% identity with the sequence Seq ID No 1 or Seq ID No 4. Thus, proteins with at least 80, 90, 95, 98, 99, 100% identity are defined as variants of human Alk-Smase. As used herein, variants of human Alk-Smase include modifications of human Alk-Smase, as well as parts of human Alk-Smase. One part of human Alk-Smase is Seq ID No 6, as being a part of Seq ID No 1 and Seq ID No 4. Parts of human Alk-Smase may further be any part comprising the active site, i.e., Seq ID No 3 in the present invention, of human Alk-Smase. The active site is described in further detail below. Further, the active site may be modified so as to achieve at least the same but preferably an increased activity of the human Alk-Smase enzyme. Such modifications are preferably done with site directed mutagenesis and further described in detail below.

Furthermore, a nucleotide sequence encoding the protein according to the invention, i.e., human Alk-Smase, is disclosed.

In one embodiment, the nucleotide sequence is a nucleotide sequence comprising the sequence Seq ID No 2.

Furthermore, a recombinant expression and secretion vector comprising a polynucleotide encoding a secretion signal peptide; a DNA sequence which promotes transcription located upstream from the polynucleotide encoding the secretion signal peptide; a DNA sequence encoding a protein according to any of the sequences disclosed of the present invention in a translation reading frame with said polynucleotide encoding the secretion signal peptide; and a transcription terminator sequence located downstream from the DNA sequence encoding said protein. Said recombinant expression and secretion vector may be used for both pro- and eucaryotic expression systems.

Also disclosed is a host cell comprising the recombinant expression system according to the invention, from which Alk-Smase is expressed. The host cell may be bacteria, a mammalian cell such as, e.g., CHO cells or Cos-7 cells, or a yeast cell.

In one embodiment, the host cell according to the invention is a mammalian cell, e.g., a CHO or Cos-7 cell, which, in the absence of the recombinant expression system according to the invention, does not normally produce an Alk-Smase.

Furthermore a method for isolation of human Alk-Smase protein is disclosed.

The method comprises the steps of

vi) providing a small intestinal or colon content from a human,
vii) homogenising the small intestinal or colon content,
viii) purifying Alk-Smase using DEAE Sephadex chromatography,
ix) purifying using Uno anion exchange chromatography,
x) purifying using hydrophobic chromatography,
   thereby isolating the human Alk-Smase protein.

Still furthermore a method for preparation of human recombinant Alk-Smase protein is disclosed. The method for preparation of human recombinant Alk-Smase protein capable of hydrolysing sphingomyelin, comprises the steps of
v) providing a host cell according to the invention and a host cell growth medium,
vi) preparing a host cell culture;
vii) culturing the host cell culture and
viii) harvesting the host cell culture and recovering the human recombinant Alk-Smase.

In a further embodiment, the Alk-Smase protein is recovered either from the culture medium, the host cells or after separating the host cells from the culture medium.

Furthermore, isolated human Alk-Smase protein is disclosed. The enzyme comprises the protein with an amino acid sequence as disclosed herein, having an active site according to Seq ID No 3 or a variant thereof.

In one embodiment the isolated Alk-Smase according to the invention is a variant, including a modified form of human Alk-Smase. The variant or modified form of human Alk-Smase may be, e.g., modified by e.g. site-directed mutagenesis to change, i.e., increase, the activity of the active site of human Alk-Smase. The activity may also, after a mutation, be the same activity as in a human Alk-Smase not having a mutated active site.

Furthermore, a composition comprising a protein according to the invention, or a nucleic acid according to the invention, or a human isolated or recombinant Alk-Smase according to the invention, and a biocompatible carrier or additive is provided. Such compositions are described in further detail below.

Furthermore, uses of a protein according to the invention, or a nucleic acid according to the invention, or a human isolated or recombinant Alk-Smase according to the invention, for the preparation of a pharmaceutical composition for the treatment of, e.g., colon cancer, are disclosed and described in further detail below.

Human Isolated Alk-Smase

The present inventors have isolated and characterized a fifth group of Smase called human Alk-Smase which has been characterized and purified despite severe difficulties in purifying the enzyme due to the nature of the enzyme and the complex proteolytic and chemical environment in which it is found. The Alk-Smase activity may influence cell differentiation, tumour growth and inflammation.

The enzyme is produced in the gut mucosa and is a constituent of the brush border membrane of the small intestine and colon but is also released into the gut lumen.

Activation of Smases may be elicited by a number of agonists known in the art. Alk-Smase of the gut may thus generate messengers that influence cell differentiation, tumour growth or inflammation.

Sequencing of the enzyme has now revealed that it differs from known acid, neutral and bacterial Smases. There are no significant homologies to these known Smases that indicate identical mechanism of action. Instead the homology searches reveal homology to the alkaline nucleotidase/pyrophosphatase (NPP) family presently comprising NPP1, NPP2, NPP3, NPP4 and NPP5 (Gijsbers et al J Biol Chem 2001; 276:1361-68).

The NPPs are a family of ectoenzymes having a number of biological effects on cellular functions by hydrolysing ATP, ADP, AMP and other nucleotides. It is now known that NPP5 is most closely related to Alk-Smase.

Smases generally generate ceramide that is further converted extra- and/or intracellularly to other sphingolipid messengers such as sphingosine (Sph) and sphingosine-1-phosphate (S-1-P). Said messengers may participate in, e.g., cell signalling.

In the gut, formation of ceramide and free sphingoid bases from dietary sphingolipids is generated by the action of Alk-Smase and B-Cer and by mucosal lactase phlorizine hydrolase known to act on glycosylceramides. The relative concentrations of sphingolipid metabolites formed will depend on the relative concentrations of enzymes generating ceramide and on their ceramidase activity on the amount of substrate available and on other conditions such as bile salt concentration and pH. Thus, by controlling the activity of Alk-Smase the relative concentration of sphingolipids may be controlled.

It has been shown that Alk-Smase and intestinal ceramidase catalyse the sequential hydrolysis of sphingomyelin, first to ceramide and phosphocholine by Alk-Smase and then to sphingosine (free sphingoid bases) and free fatty acid. It has also been shown that the free sphingoid bases are well absorbed and metabolised in the gut thus generating ceramide and sphingosine-1-phosphate after absorption.

Identification of Human Alk-Smase and its Active Site

The present invention provides purified and sequenced human Alk-Smase protein and the identified gene coding for this protein.

The present invention demonstrates that Alk-Smase has no significant homology to known acid, neutral or bacterial Smases. It is a member of the alkaline nucleotidase family. Alk-Smase has a characteristic active site sequence reading AFVTMTSPCHFTLVTGKY (Seq ID No 3).

Alk-Smase is closely related to nucleotidase/pyrophosphatase5 (NPP5).

The present invention includes compositions comprising recombinant protein using sequences disclosed in the present invention, as well as modifications and parts thereof. Such modifications and parts thereof are described in further detail below.

The present invention further includes a composition comprising human Alk-Smase and modifications thereof and optionally further comprising B-cer or lactase-phlorizin hydrolase as well as substrates for these enzymes. Such substrates are known in the art.

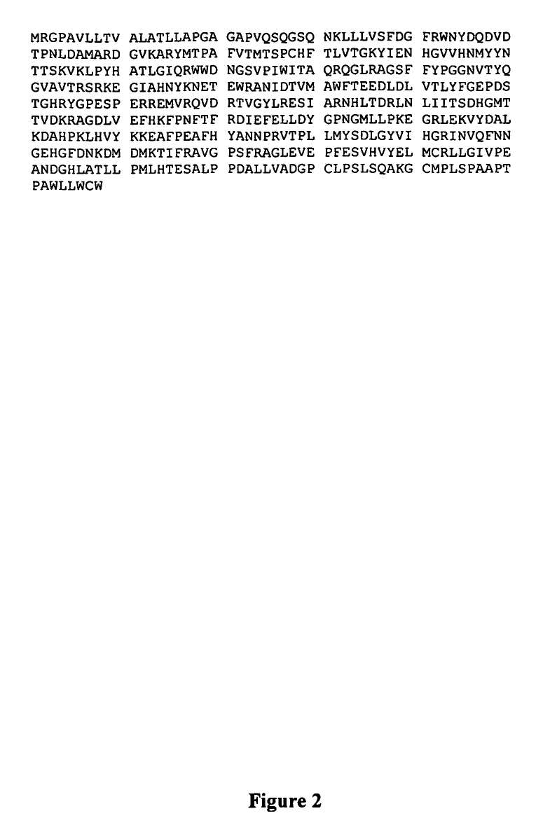

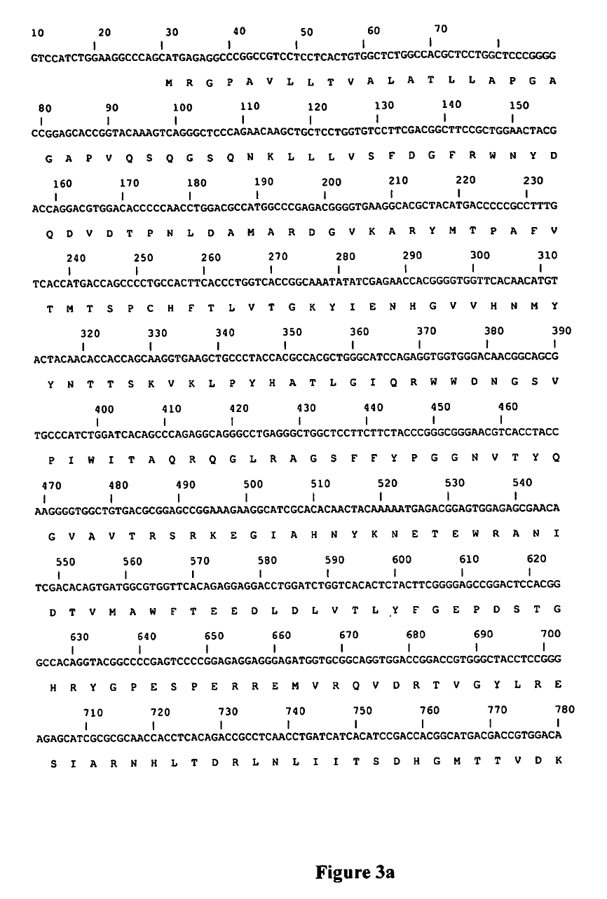

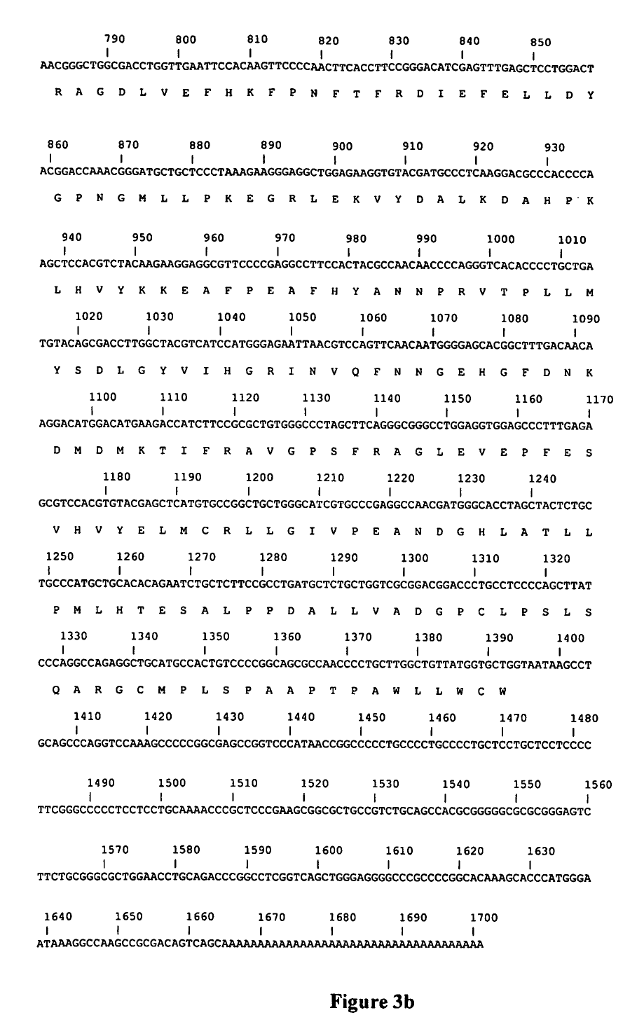

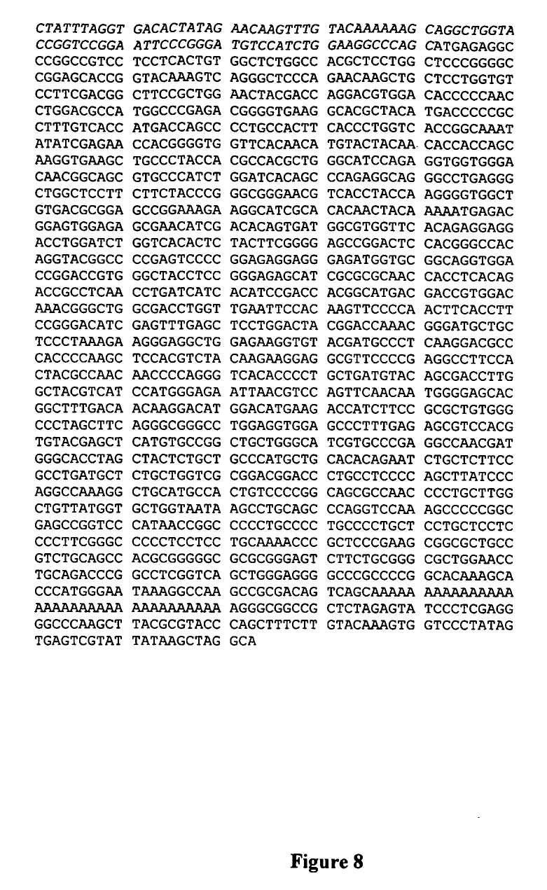

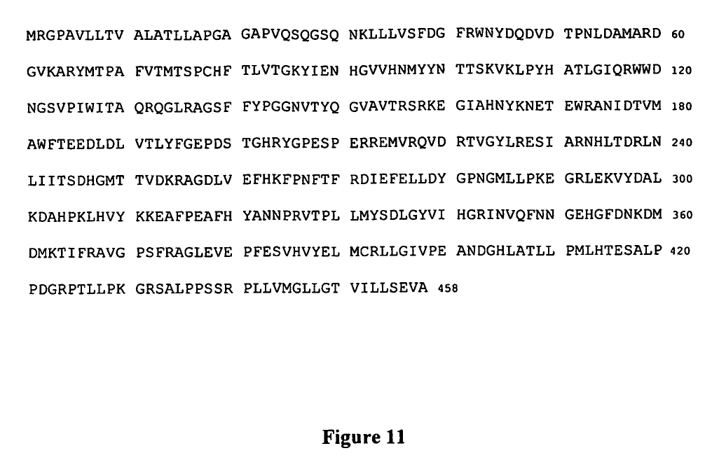

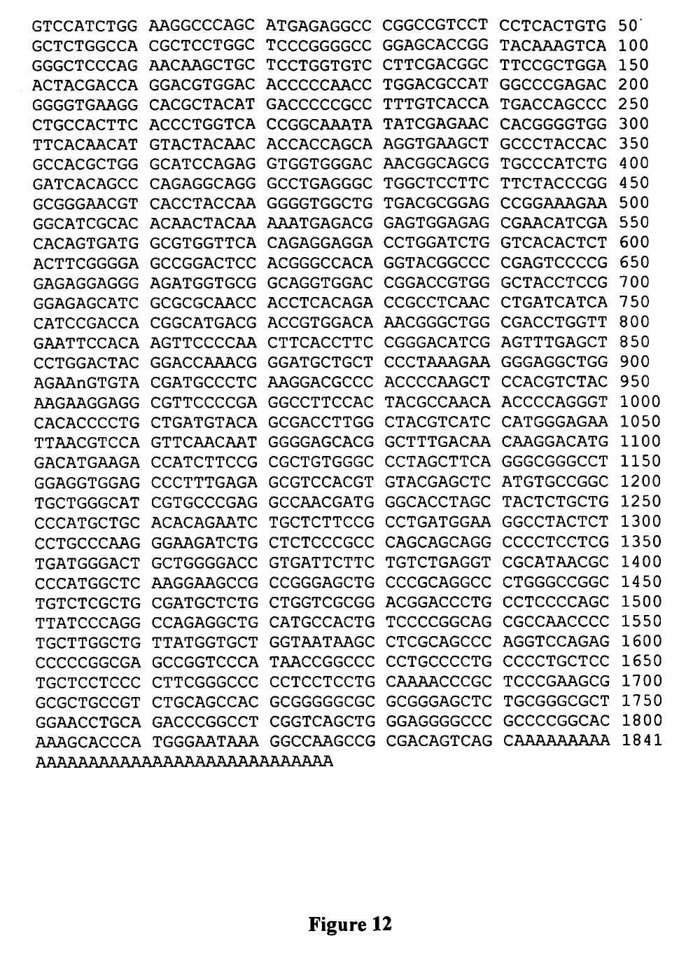

TABLE 1
Sequence ID number

| Seq ID No | Aa no | Nt no | Name of sequence | comment |
|---|---|---|---|---|
| Seq ID No 1 | 1-458 | — | Alk-Smase - | |
| | | | variant1 | |
| Seq ID No 2 | — | 10-1700 | cDNA Alk-Smase - | Shown in FIG. 3 and 8 |
| | | | variant1 | Nt10-30 = not translated |
| | | | | region |
| | | | | Nt31-1404 = reading frame |
| | | | | Nt1407-1676 = not |
| | | | | translated region |
| | | | | Nt1676-1701 = polyA tail |
| Seq ID No 3 | 70-87 | — | Active site1 of Alk- | |
| | | | Smase | |
| Seq ID No 4 | 1-458 | — | Alk-Smase - | Shown in FIG. 11 |
| | | | variant2 | |
| Seq ID No 5 | — | 1-1878 | Alk-Smase - | Shown in FIG. 12 |
| | | | variant2 | Nt1-20 = not translated |
| | | | | region |
| | | | | Nt21-1394 = reading frame |
| | | | | Nt 1395-1841 = not |
| | | | | translated region |
| | | | | Nt1842-1878 = polyA tail |
| Seq ID No 6 | 1-415 | — | Alk-Smase - | Shown in FIG. 2 and 11, as |
| | | | variant3 | fragment of sequence |
| | | | | displayed |
| Seq ID No 7 | 71-80 | — | Active site2 of Alk- | |
| | | | Smase | |

Purification of Alk-Smase

Human Alk-Smase has now been purified from small intestinal content and its' sequence obtained by MALDI-TOF spectrum and micro Edman degradation. Alk-Smase is specifically expressed in the small intestine and colon and participates in the digestion of dietary sphingomyelin. It is down regulated in colonic tumours and in familial polyposis, and may generate anticarcinogenic sphingolipid messengers in the gut. The enzyme is located to the brush border and in part released into the lumen.

The cDNA has also been cloned and found to match the amino acid sequence.

Proteins with a high degree of homology have been identified in the mouse and the rat.

Thus, a method for purification of human Alk-Smase protein is disclosed.

The method comprises the steps of

xi) providing a small intestinal or colon content from a human,
xii) homogenising the small intestinal or colon content
xiii) purifying Alk-Smase using DEAE Sephadex chromatography
xiv) purifying using Uno anion exchange chromatography,
xv) purifying using hydrophobic chromatography,
   thereby isolating the human Alk-Smase protein.

The human Alk-Smase has been purified by a combination of DEAE Sephadex chromatography, Uno anion exchange chromatography and hydrophobic chromatography.

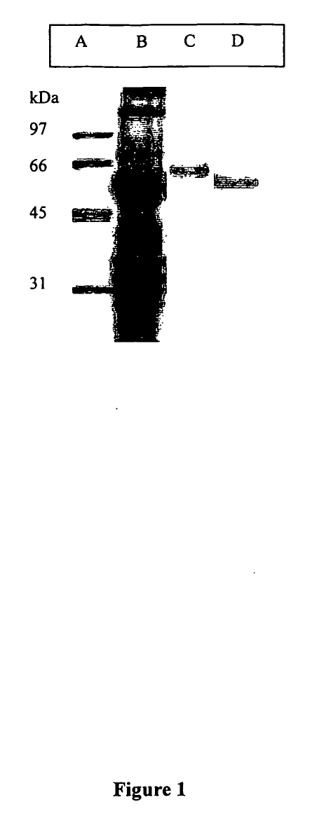

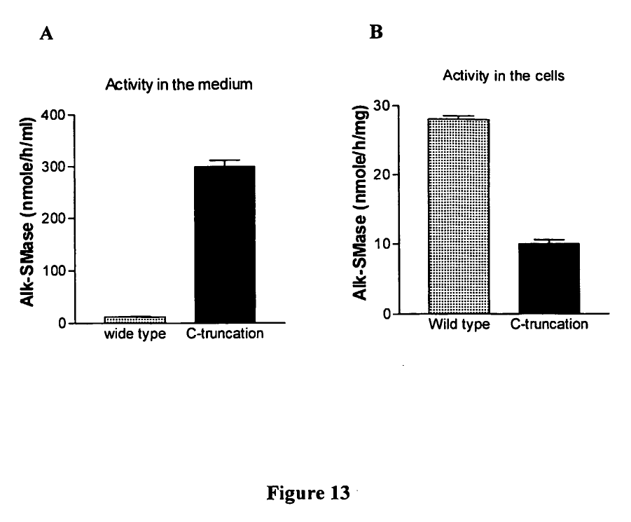

The obtained protein has a molecular weight of 58 kD as seen in FIG. 1. Structural analysis of the protein by MALDI-TOF and micro-Edman degradation reveals a polypeptide of 415 amino acids, and the amino acid sequence seen in FIG. 2. This sequence comprises a sequence necessary for the protein to exhibit Alk-Smase activity as indicated by the findings presented in FIG. 13. The figure shows that CHO cells transfected with cDNA encoding amino acid 1-415 have high Alk-Smase activity. The cells also secrete large amounts of Alk-Smase into the medium. The activity of Alk-Smase produced was higher than in CHO cells transfected with cDNA containing the full sequence, i.e., Seq ID No 5 (FIG. 13). The invention thus discloses that any protein comprising the sequence derived from the analysis and disclosed herein, variants or parts thereof, of purified human intestinal Alk-Smase has Alk-Smase activity although it may contain different C-terminal sequence(s). Specifically, Seq ID No 3, the active site, is the most important for the Smase activity and must, thus, be included in a sequence according to the invention for preserving Smase-activity.

Since the full amino acid sequence has several tryptic cleavage sites, particularly in the C terminal from 416-458 there are 3 tryptic cleavage sites, it is furthermore included that the Alk-Smase purified from human intestinal content may have undergone such cleavage during its release from the brush border into the lumen.

Further disclosed is also the characteristic of an active site sequence in the Alk-Smase. The active site of the enzyme comprises the sequence FVTMTSPCHF (Seq ID No 7). The disclosure of the active site of human Alk-Smase characterizes the substrate specificity and furthermore the biological effects of the Alk-Smase activity.

The present invention discloses the amino acid sequence and the full length cDNA sequence of human intestinal Alk-Smase.

The human Alk-Smase is a 458 amino acid protein related to the nucleotidase/pyrophosphatase (NPP) family and is coded for by a gene located on chromosome 17. The human Alk-Smase further comprises six exons.

In contrast to the NPPs, the enzyme is not stimulated by divalent metal ions. The enzyme was further inhibited by Zn²⁺.

Sequence alignments indicated the presence of an active site region sequence FVTMTSPC (Seq ID No 8) in which the middle T corresponds to a crucial Thr that undergoes reversible phosphorylation in the conserved PTKTFPN (Seq ID No 9) active site sequence of known NPPs (Gijsbers et al J Biol Chem 2001; 276:1361-68).

Thus, Alk-Smase is a novel protein related to the NPP family but with specific features that may be essential for its Alk-Smase activity.

Methods for Determining the Amino Acid Sequence of Human Alk-Smase

The band corresponding to the purified Alk-Smase is shown in FIG. 2. The band was cut and extracted with techniques known in the art. After digestion with trypsin the fragments were separated by HPLC and analysed by MALDI-TOF and micro Edman degradation (P Edman, G Begg G. Eur J Biochem 1:80-91, 1967, J R Yates et al Anal Biochem 1993; 214:397-408 and A P Jonsson et al Anal Bichem 2001; 73:5370-7, Oppermann, M., Cols, N., Nyman, T., Helin, J., Saarinen, J., Byman, I., Toran, N., Alaiya, A. A., Bergman, T., Kalkkinen, N., Gonzàlez-Duarte, R. & Jörnvall, H. (2000)).

Identification of foetal brain proteins by two-dimensional gel electrophoresis and mass spectrometry is performed as outlined below. Comparison of samples from individuals with or without chromosome 21 trisomy was performed as previously described (Eur. J. Biochem. 267, 4713-4719, Bergman A.-C., Oppermann, M., Oppermann, U., Jörnvall, H. & Bergman, T. (2000)). Characterization of gel separated proteins was performed as previously described (Proteome and Protein Analysis (Kamp, R. M., Kyriakidis, D. & Choli-Papadopoulou, Th., eds.) Springer-Verlag, Berlin Heidelberg, pp. 81-87).

Analysis of cDNA Sequence

Materials

An expressed tag (pCMV-Sport6, Clone ID: IMAGE 5186743) was obtained from ResGen (Huntsville, Ala., USA). Human multiple tissue Northern blots, human digestive system Northern blot, Zoo-Blot which contains 9 different species genomes and the Express Hyb solution were purchased from Clontech, Palo Alto, USA. All primers were purchased from DNA technology (Aarhus, Denmark). [³²P]dCTP was purchased from Amersham Pharmacia (Freiburg, Germany).

Cloning Alk-Smase Full-length cDNA

A novel partial cDNA sequence (415 amino acid residues) coding Alk-Smase was obtained by the microdigestion amino acid analysis. After searching different public databases, no homologous protein sequence was identified.

Oligonucleotide primers based on the sequence of micro-digested amino acid sequence analysis together with the EST database were used to clone the Alk-Smase. The 5′ and 3′ ends of the Alk-Smase cDNA were amplified from the human small intestine library and a contiguous 1700 nucleotide sequence was subsequently amplified by using 5′ and 3′ Alk-Smase cDNA ends as templates. A complete cDNA and translated protein sequence of Alk-Smase is shown in FIG. 3.

A human expressed sequence tag (clone ID: IMAGE 5186743) was found identical to the obtained amino acid sequence. Based on this expressed tag sequence, two oligonucleotides, oligo-1 and oligo-2, corresponding to the sense 5′-GGCCCAGCAT GAGAGGCCCG GCCGTCC (Seq ID No 10) and antisense 5′-GGACGGCCGG GCCTCTCATG CTGGGCC (Seq ID No 11) were synthesized. A human small intestine 5′-stretch plus cDNA library was used as template in the PCR amplification.

A PCR reaction (50 μl) was performed in a buffer of 25 mM KCl, 10 mM Tris-HCl, pH 8.85, 0.05% Triton X-100, each dNTP at 0.2 mM, each primer at 0.5 μM, 2.5 mM MgCl₂, and 2.5 units of Pwo DNA polymerase using 30 temperature cycles of 94° C. (1 min), 65° C. (1 min), and 72° C. (3 min). In the first two PCR reactions, oligo-1 was combined with a vector-specific sequencing primer, P1-TAATACGACTCACTATAGGG (Seq ID No 12), and oligo-2 with the reverse sequencing primer, P2-TCCGAGATCTGGACGAGC (Seq ID No 13).

The PCR products were combined in a third PCR using primers P1 and P2 to obtain full-length cDNA, which was then sequenced on both strands using a sequencing kit from PE Applied Biosystems. The sequence experiments were repeated at least three times.

The Alk-Smase cDNA contained a 1377 nucleotides coding sequence with 20 nucleotide of 5′ untranslated region and 267 nucleotide 3′ untranslated region except for the poly(A) sequence.

In the open reading frame coding for Alk-Smase, 61.7% of the nucleotides are either G or C. Both 5′ untranslated region and 3′ untranslated region are rich in GC residues (65% of 20 nucleotides in 5′ untranslated region) and (71.9% of 267 nucleotides in 3′ untranslated region), respectively.

The predicted amino acid sequence of the open reading frame contained 458 amino acids and is shown in FIG. 3a and b and in FIG. 11. FIG. 11 shows the results from repeated analyses in which the last 36 residues from 423 to 458 at the C-terminal have or have not been included (Duan et al J Biol Chem 2003; 278:38528-36). As shown in FIG. 13, these 36 residues are not essential for Alk-Smase activity and the released enzyme in the intestinal lumen in vivo may also lack this domain. Both analyses are based on clone ID IMAGE 5186743, identified as the gene coding for Alk-Smase.

Northern and Southern Blotting Analyses

A 439 bp DNA fragment of Alk-Smase was amplified by PCR using primers 5′-GGCCCGAGAC GGGGTGAAGG CACGCTACAT GACCCCCGCC (Seq ID No 14) and 5′-TGGCCCGTGG AGTCCGGCTC CCC (Seq ID No 15). The DNA fragment and a control probe (G3PDH, purchased from Clontech) were radiolabeled with [³²P]dCTP using the random primer method (RediPrime; Amersham Pharmacia Biotech, Uppsala, Sweden) to specific activities of 3-7×10⁸ cpm/μg.

Human multiple-tissue Northern-blotting membrane containing mRNA from 12 different organs, human digestive system Northern blot membrane and Zoo blots membrane were hybridized with radiolabelled probes. Hybridizations and washings were carried out at stringent conditions. Hybridisation was performed at 50° C. in a hybridization solution (Clontech) with ³²P-labeled DNA probes. The blot was washed several times in 2×SSC/0.1% SDS solution at room temperature for 2 hrs and twice in 0.1×SSC/0.05% SDS at 50° C. for 40 min. The washed blot was exposed to X-ray film at −70° C. from 1-3 days. The autoradiographs were analyzed with a scanner (Epson-1600).

The membrane was stripped with boiled water in the presence of 0.5% SDS for 10 min and then rehybridized with the control probe. The relative mRNA levels were calculated with a Macintosh computer using the software of Quantity One (Version 4.2.1, Bio-Rad Laboratories) and presented as volume (intensity×mm²).

The Amino Acid Sequence of Human Alk-Smase

The description for the determination of the amino acid sequence is given above.

The amino acid sequence of the purified Alk-Smase is shown in FIG. 2.

Characterization of Alk-Smase

A subsequent characterization of human Alk-Smase gave the following characteristics:
the pH-optimum for Alk-Smase is around 8.5
the enzyme requires bile salts
the enzyme is stimulated more efficiently by conjugated cholic acids than by other bile salts
the enzyme is extremely resistant to trypsin and chymotrypsin in its undenatured form. It is not inhibited by EDTA and does not depend on magnesium or Zn2+ ions for its action

When expression or existence was evaluated in different species the enzyme activity was found in rat, mouse, pig, baboon, sheep and dog but not in guinea pig. It was also found in germ-free mice. It was found missing only in guinea pig. Subsequent studies have, however, indicated that this enzyme is also from bile.

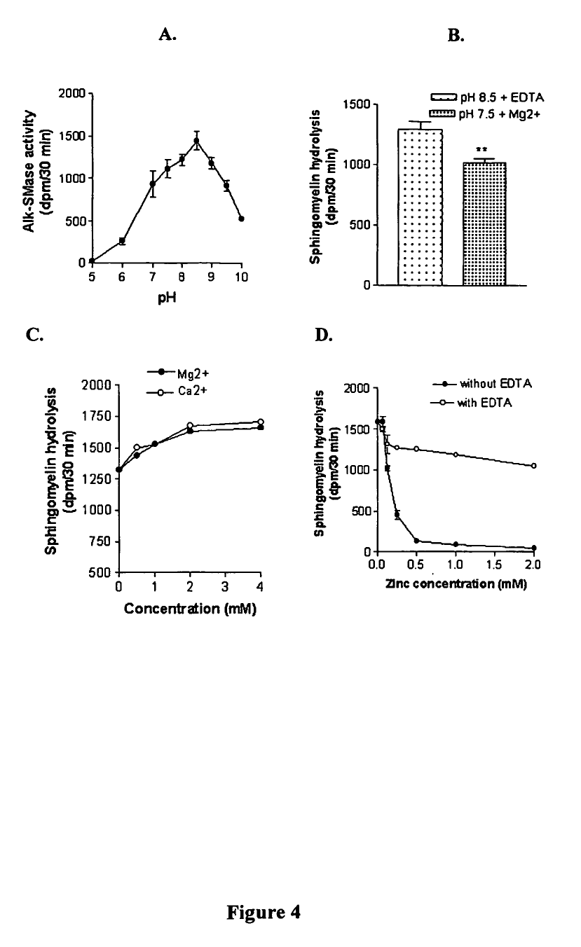

The properties of Alk-Smase were examined with special attention to those that distinguish the enzyme from acid and neutral Smases. FIGS. 4a-c show the characteristics of human Alk-Smase. FIG. 4a shows the optimal pH of the enzyme. The activity was low at pH less than 6 and sharply increased when pH was 7 or higher. The maximal activity was obtained at pH 8.5, the activity at pH 7.5 being about 68% of the maximal activity.

To distinguish whether the enzyme was different from the Mg dependent neutral Smase, activity at pH 7.5 in the presence of 4 mM Mg2+ in comparison wit pH 8.5 in the presence of 2 mM EDTA was assayed. As shown in FIG. 4b, the activity at alkaline pH without divalent cations was significantly higher than at 7.5 with Mg present. The dependency of the enzyme on Mg2⁺ and Ca2⁺ was then studied. Those studies showed that the activity was slightly increased with increasing concentrations of Mg and Ca2⁺. However relatively high activities were detected under Ca2⁺ and Mg2⁺ free conditions as shown in FIG. 4c. FIG. 4d shows that Zn2⁺, which can activate other types of Smases in serum and in the arterial wall, significantly inhibited Alk-Smase activity with a 50% inhibition at 0.015 mM. Most of the inhibitory effect of Zn2+ was reversed by 2 mM EDTA.

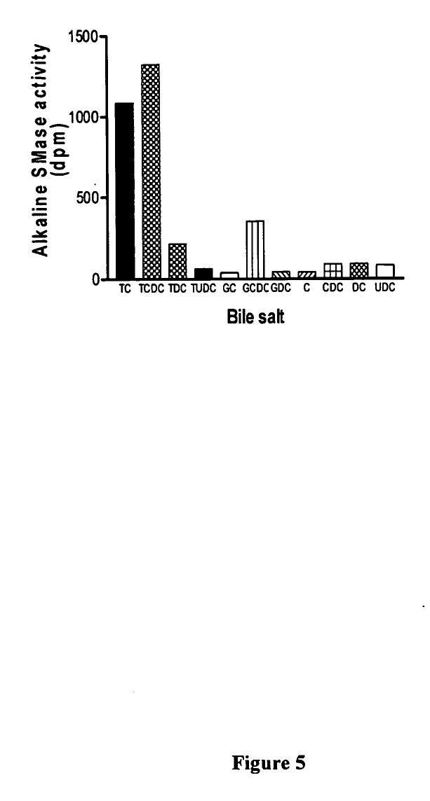

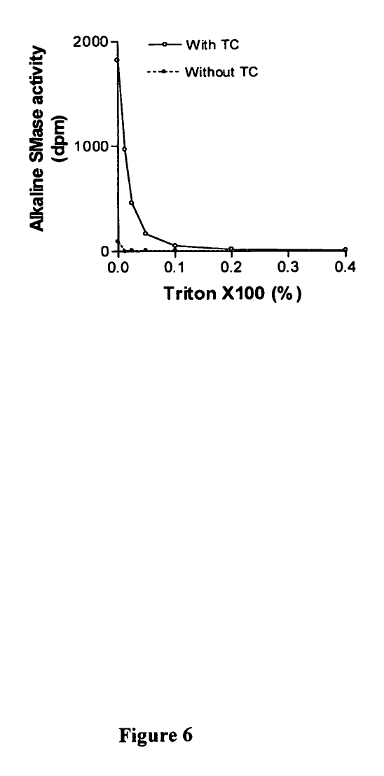

As bile salts are important factors for lipid digestion, the effects of different bile salts on human Alk-Smase were investigated. Of eleven examined bile acids, all stimulated Alk-Smase activity, the concentration dependence exhibiting a bell shaped curve with maximum at the CMC for each bile salt. FIG. 5 shows effects of bile salt on human Alk-Smase. The activities were determined in the presence of different concentrations of bile salts. The maximal stimulated effects of each bile salt are shown in the figure. However, when the maximal effects of the bile salts were compared, taurocholate (TC) and taurochenodeoxycholat (TCDC) were far more effective than other bile salts as shown in FIG. 5. The glycine conjugated bile salts were less potent than the taurine conjugated ones. CHAPS, which has the identical nucleus of the TC but a different side chain structure, only slightly increased the activity at very low concentration (0.025 mM) but blocked the stimulatory effect of TC as shown in FIG. 6, left panel. Triton X 100, which has been widely used for assaying both acid and neutral Smase, strongly inhibited human Alk-Smase activity in the presence or absence of TC as shown in FIG. 6, right panel. The figure shows Alk-Smase activity in the presence of different concentrations of Triton X 100 with and without taurocholate (10 mM). The figure further shows that Triton X 100 dose dependently inhibits human Alk-Smase.

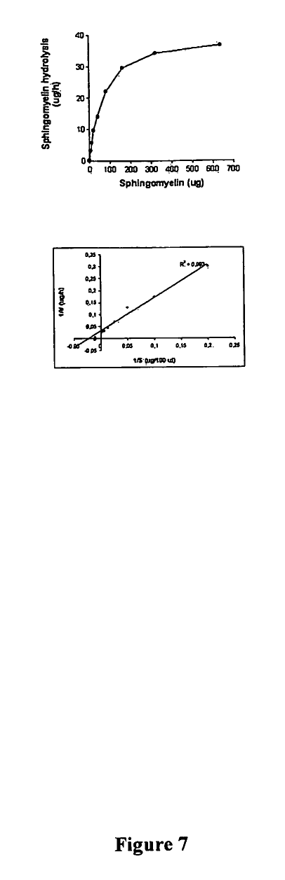

Glutathione was previously found to inhibit mammalian neutral Smase. In the experiment, the inhibitory effect of glutathione on both Alk-Smase and bacterial neutral Smase activities was compared. As shown in FIG. 7, the reduced form of glutathione sharply abolished the activity of neutral Smase at concentrations higher than 5 mM, but only slightly reduced the activity of Alk-Smase. At 7.5 mM glutathione the activity of neutral Smase was reduced by 98%, but Alk-Smase was reduced by only 1%. The oxidized form of glutathione had no effect on the neutral or the alkaline Smase (data not shown). The hydrolytic capacity of the enzyme was examined by incubating 5 ng Alk-Smase with SPHINGOMYELIN masses ranging from 5 to 640 micrograms in 100 microliters assay buffer. A shown in the Lineweaver-Burk plot in FIG. 7, 1 mg of the enzyme is able to hydrolyse about 11 mmole SPHINGOMYELIN in one hour under the assay conditions presented.

Interpretation of the Structure

The amino acid sequence is shown in FIG. 2 (Seq ID No 1).

The cDNA sequence is shown in FIG. 8 is from nucleotide 92-1735 without poly A tail (Seq ID No 4). Nucleotides 1-91 and those after poly A are from the vector.

An initial BLAST search indicated that the enzyme exhibited homology to the NPP family but not with phospholipase C or neutral and acid Smases. The identity with the members of the NPP family is about 30 to 35%. After the amino acid sequence was obtained, subsequent BLAST searches including all non-redundant GenBank identified the three recently submitted sequences gi|273712361|, gi|27690846| and gi|28515289|. Gi|27371236| is a clone submitted from NIH mammalian gene collection, derived from a pooled colon-kidney-stomach library. It codes for 464 amino acids and exhibits 100% homology with Alk-Smase for the 422 amino acids counted from amino acid 7 to 430·gi|27690846| is a direct submission of a predicted rat protein and gi|28515289| is a direct submission of a predicted mouse protein. Both are 83% identical with human Alk-Smase.

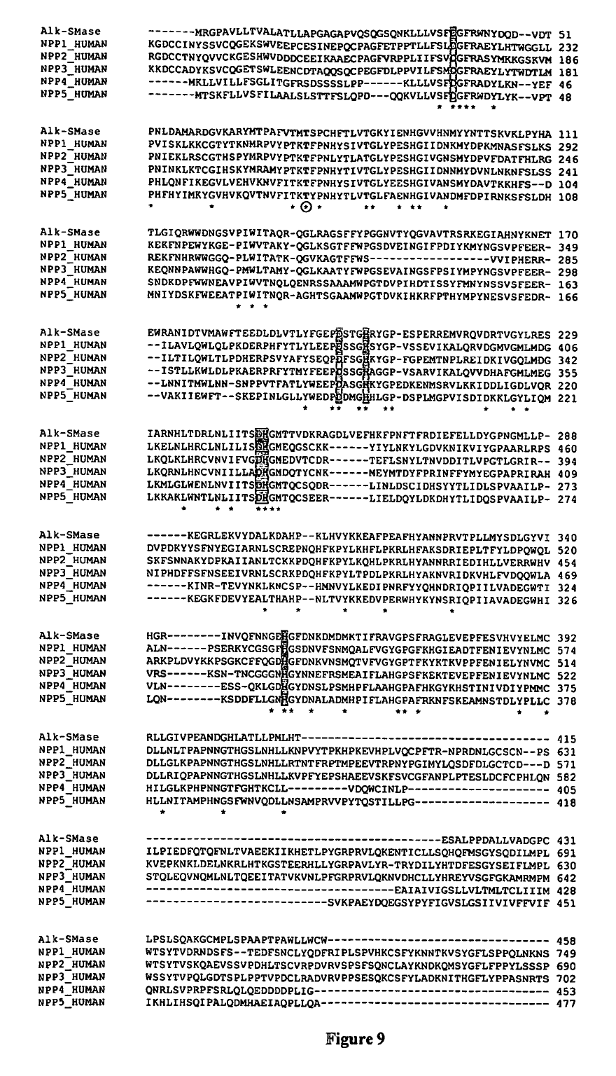

The sequence alignment of Alk-Smase with NPP 1-5 is shown in FIG. 9. The sequence starting with amino acid 32, i.e., KLLLVSFDGFRWNYD (Seq ID No 16) exhibited homology to all the NPPs. The function of this region is not known. The catalytic residue of NPPs is Tyr as marked with * in the figure. This residue is conserved in Alk-Smase. The amino acids in adjacent to the Tyr form an active site region which is important for substrate specificity (Gijsbers et al JBC 2001:276-1361-8). This active site region in Alk-Smase has been modified as shown in the figure. K is replaced by M, F is replaced by S, and N is replaced by C. Notably the three most similar clones mentioned above all contain the same potential active site region as Alk-Smase.

Metal Binding Sites

According to the conserved site three dimensional structure model for NPP1 of Gijsbers et al (JBC 2001;276:1361-68) D358, H362, H517, as well as D405, H406, and D200 are important residues to form metal coordinating sites. All these amino acids are conserved in Alk-Smase as shaded in FIG. X. According to Gijsbers et al (J Biol Chem 2001; 276:1361-68) the metal ions seem to stabilise the conformation needed for hydrolysis of the water soluble phosphate esters rather than participate directly in the reaction mechanism. If this is correct, interaction with Zn ions may inhibit the hydrolysis of Sphingomyelin because one more conformation of the catalytic region of the protein is needed for Sphingomyelin hydrolysis than for hydrolysis of nucleotides.

Use of Alk-Smase

Human and rat alkaline Smase inhibit proliferation of the human colon cancer cell line HT 29 in cellular experiments. For further details see Experiment 1. Thus, one use of human Alk-Smase is to inhibit colon cancer. In one embodiment, Alk-Smase is used to prepare a pharmaceutical composition for inhibition of colon cancer. Compositions are described in further detail below in the present invention.

By analyzing enzyme activity in colon tumours and in surrounding mucosa it has been found an average decrease in the Alk-Smase activity in the tumour of 70% (E Hertervig et al Cancer 1997; 79:448-53). In patients with familial adenomatous polyposis the activity was reduced by about 90% compared to normal mucosa (E Hertervig et al Br J Cancer 1999; 81:232-6). Thus, one use of Alk-Smase is to provide recombinant human Alk-Smase, or the composition mentioned above, in a therapeutic effective dose to patient in the need thereof, such as a human being with colon tumours.

Function and Uses of Alk-Smase

The intestinal Alk-Smase may have several functions. Undoubtedly it has an important role in the digestion of dietary sphingomyelin, and is expressed in the intestine of the newborn just before birth (J Lillienau et al Lipids, 2003, 38:545-9), i.e., just before the ingestion of milk that contains Sphingomyelin as one of the major polar lipids.

The strong and selective activation of the enzyme by taurocholate and taurodeoxycholate (Y Cheng et al J Lipid Res 2002; 43:316-324) suits this function well, and also indicates that bile salt stimulation is not only a physicochemical effects, since all the examined bile salts efficiently form mixed micelles with the Sphingomyelin at the substrate concentration used. There must thus be either a specific interaction of the most efficient bile salts with the enzyme, which influences conformation or conformational stability of the enzyme, or a specific orientation of the polar OH and taurine groups in proximity to the polar phosphocholine head group of Sphingomyelin, that determine substrate specificity.

Other functions for human Alk-Smase may be that the enzyme may generate bioactive sphingolipid metabolites also from endogenous substrates.

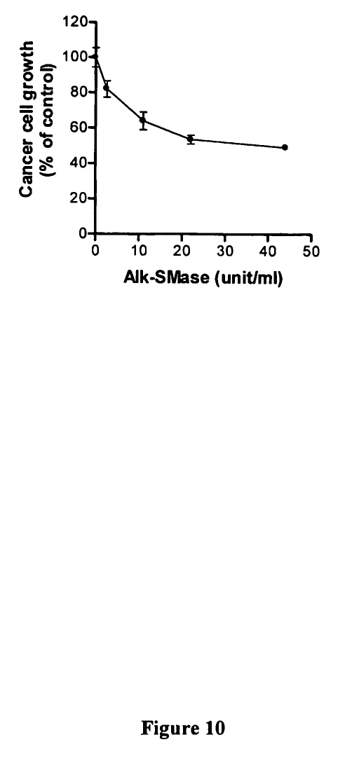

Other studies show that Alk-Smase may influence tumour growth. Adding either human or rat Alk-Smase to HT-29 colon carcinoma cells in culture was found to inhibit cell growth and decreased DNA synthesis as shown in FIG. 10 and in experiment 1 (E Hertervig et al J Cancer Res Clin Oncol 2003; 129:577-82). FIG. 10 shows the effect of rat Alk-Smase on proliferation of colon cancer cells. HT29 human colon cancer cells were incubated in RMPI-1640 medium with L-glutamine, containing antibiotics and 10% (v/v) heat inactivated fetal calf serum. At the exponential growth phase, the cells were incubated with purified rat Alk-Smase at different doses for 18 h. The cell proliferation rates were measured by WST reagent. Results are Mean± SD of three individual duplicate experiments. The figure shows that Alk-Smase dose-dependently inhibited cell growth.

Human Alk-Smase may also generate bioactive sphingolipid metabolites from endogenous substrates. Other ectoenzymes have significant biological activities and since Alk-Smase might be secreted both into the gut lumen and into the lymphatic space of lamina propria one may ask if Alk-Smase may have other functions as well, mediated by its actions on e.g. epithelial, stromal and immunocompetent cells.

The digestion or intracellular hydrolysis of sphingomyelin generates sphingolipid messengers which regulate cellular functions.

The enzyme may further counteract cell proliferation as shown in example 1. The enzyme activity is lowered in colon tumour and in familial adenomatous polyposis. It is therefore of great importance to be able to regulate Alk-Smase activity for therapeutic purposes of, e.g., colon tumour and in familial adenomatous polyposis.

The enzyme may be used in formulations together with substrates that may generate biologically active compounds in the colon, e.g., short chain sphingomyelin.

The present invention further discloses the use of disclosed amino acid sequences to make possible the use of the human Alk-Smase, or variants including modified Alk-Smase, or parts thereof, with a specific defined active site in clinical use and for preparing knockouts and transfection studies.

Variants of human Alk-Smase may be modified Alk-Smase and include human Alk-Smase with a mutated active site. The mutations may give the same or an increase in activity of human Alk-Smase. The increase may be in activity as compared to a non-mutated human Alk-Smase enzyme.

Discussion

Disclosed herein is the isolation and identification human intestinal Alk-Smase as a novel protein related to the NPP family, but with only a 30-36% identity to the known NPP1-4. Earlier studies have only partly purified and characterized Alk-Smase based on its activity from rat and humans, and prepared antisera based on this partly purified enzyme.

Attempts to obtain the full sequence and clone the enzyme have met severe difficulties due to the nature of the enzyme.

In the present invention mass fragmentographic analysis and micro Edman degradation were combined to obtain a sequence that matched the sequenced part of a cDNA clone derived from pooled material of human colon, lung and kidney (expressed tag pCMV-Sport6, Clone ID: IMAGE 5186743), which was fully sequenced and found to match the peptide sequence. During the late course of the study, clone gi|27371236| appeared in the GenBank containing a sequence with a 100% match of overlapping parts with the enzyme. This clone has been derived from a cDNA library from colon plus kidney and stomach and is described as an NPP-like protein with unknown function. Two recently submitted rodent clones match the enzyme closely. The Riken mouse full length cDNA clone gi|27690846| has 83% identity, and the predicted rat gene gi|20914245 has 83% identity with human Alk-Smase. The mouse and rat sequences code for sequences of 450 (rat) and 427 (mouse) amino acids.

Sequence alignments demonstrate homologies between Alk-Smase and several conserved regions in the NPPs. The structural and catalytic similarities between NPPs and APs were recently analyzed by Gijsbers et al. The catalytic region of the NPPs is highly conserved and contains the crucial Thr that undergoes reversible phosphorylation during the reaction.

In both human Alk-Smase and the postulated rat and mouse Alk-Smase the corresponding sequence is TMTSPC. It is thus suggested that Alk-Smase is likely to involve an active site including this Thr, the catalytic site being modified to serve the substrate preferences of the enzyme. In line with the computational analysis performed by Gijsbers et al for NPP1 a similar analysis on human Alk-Smase indicated that the three dimensional folding of this region in the Alk-Smase may be similar to that of crystalline E coli AP (alkaline phosphatase).

A difference between Alk-Smase and NPPs is that NPPs are generally stimulated by divalent metal ions, the activity being increased by Zn2+, Mg2+ and Ca2+, and distinct metal co-ordinating regions have been identified. Sequence alignments identified regions in Alk-Smase likely to correspond to the D358N or H326Q in NPP1 designated as metal co-ordinating sites by Gijsbers et al (J Biol Chem 2001; 276:1361-68). Yet, no significant stimulation by divalent metal ions or inhibition by EDTA for Alk-Smase has been shown. On the contrary a distinct inhibition by Zn2+ ions was observed.

The enzyme was found to be located at the brush border of intestinal epithelial cells using the sensitive immunogold technique, and was also found in Golgi structures and endovesicles of the epithelium (RD Duan et al J Lipid Res 2003, 44:1241-50). These findings probably reflect synthesis in the epithelial cells. The question whether there is a specific incorporation into the brush border apical membrane resulting in a loose association due to the lack of a strongly hydrophobic transmembrane part, an apical secretion, or a secretion also via the basolateral membrane remains to be established. The second alternative is that synthesis occurs in other cell types in the lamina propria followed by transcellular transport accomplished by adhesion to the brush border and release into the gut lumen. Knowing the structure of Alk-Smase should lead to the clarification of the site and regulation of synthesis, and of the structural factors that determine its membrane targeting and secretion as well as its substrate specificity.

At present the most intriguing functional similarity between Alk-Smase and NPP is the earlier observation that NPP2 has lysophospholipase D activity (M Umezo-Gozo et al J Cell Biol 2002; 158:227-33). To determine whether Alk-Smase also has lysophospholipase D activity, a trace amount of ¹⁴C-palmitoyl labelled 1palmitoyl-lysosphosphatidylcholine was incubated with Alk-Smase using the optimal incubation conditions previously described (R D Duan and Å Nilsson Methods Enzymol 2000; 311:276-86). Lipids were then extracted according to known methods (Bligh and Dyer J Biochem Physiol 1959; 37:911-917), and separated on silica gel G plates that were developed in chloroform:methanol:water:acetic acid 25:20:0.3:3. The silica gel separates lysophosphatidylcholine and lysophosphatidic acid that are formed if there is a lysophospholipase D and monoglyceride formed by lysophospholipase C. It was found that 80% could be degraded to ¹⁴C-monoglyceride in one hour but formation of lysophosphatidic acid was not demonstrated. It is therefore further disclosed herein that Alk-Smase may also influence cellular functions by removing lysophosphatidylcholine which can then not be converted to lysophosphatidic acid. Lysophosphatidic acid is a metabolite that stimulates growth of colon tumour cells. Our earlier studies have shown that Alk-Smase is not a general phosphlipase C since it has low activity against phosphatidylcholine (RD Duan, A Nilsson Methods Enzymol 2000; 311:276-86).

In summary, the present invention discloses the sequence and gene structure of human intestinal alkaline Smase. The amino acid sequence disclosed confirms a similarity to the NPP family but not to acid or neutral Smase or to phospholipase C. The present invention further confirms that it is a mammalian enzyme selectively expressed in the intestine and colon and not a bacterial enzyme.

Furthermore the invention relates to an oligonucleotide sequence, which hybridises under stringent conditions (as defined above) to a nucleotide sequence and/or a nucleotide sequence molecule according to the invention.

Preparation of Recombinant Alk-Smase

The gene for Alk-Smase may be inserted into an expression vector for pro- or eucaryotic expression of the human Alk-Smase.

Examples are, e.g., expression in bacteria such as E. coli; mammalian cells such as CHO cells, or yeast cells. Transformed E. coli expressing the Alk-Smase gene may be utilized for enzyme production. Protocols for cloning methods are well known in the art (Current Protocols in Molecular Biology, Wiley Interscience and Greene (publishers), Ausubel, F. M., Brent R., Kingston R. E., Moore, D. D., Seidman, J. G., Smith J. A., Struhl, K., Sambrook et al. (1989) Molecular cloning: A laboratory manual, 2^{nd} edn. Cold Spring Harbor Laboratory Press, New York).

An embodiment for preparing recombinant human Alk-Smase in mammalian CHO cells, bacterial E coli, and yeast is outlined below.

Expression in Mammalian CHO Cells

The expression of Alk-Smase in mammalian cells may be performed by the T-REx systems®.

The Alk-Smase gene may be obtained from pCMV-sports6-Smase vector according to the invention by digesting with KpnI and Not I. The expression vector may be constructed by ligating the gene with KpnI/Not I digested pcDNA™4/TO (Invitrogen) to form pcDNA™4sm/TO vector. When the vector is transfected into CHO cells, its expression is under the regulation of another vector called pcDNA™6/TR vector (Invitrogen). The regulatory vector is provided for high-level expression of the tetracycline repressor (TetR) protein. Other similar systems known in the art may further be used. When CHO cells are co-transfected with both pcDNA™4-sm/TO and pcDNA™6/TR, the expression of Alk-Smase may simply be triggered by adding tetracycline in the cell culture medium. Further embodiments include T-Rex™-CHO cells which have been transfected with the pcDNA™6/TR vector, just transfect the cells with pcDNA™4sm/TO vector comprising human Alk-Smase. Lipofectin™2000 (Invitrogen AB), or Multiporator (Bio-Rad) may be employed for transfection. The transfected cells will be selected by selective medium (Ham's F-12K medium with 2 mM L-glutamine adjusted to contain 1.5 g sodium bicarbonate, 90%; fetal bovine serum, 10%; Zeocin™ 100 ug/ml), and plated to 60 mm plates, and cultured until Zeocin™-resistant colonies are detected. The expand clones will be seeded to 6-well plates and the expression is in this system induced by adding tetracycline to the medium.

Expression of Recombinant Alk-Smase in Mammalian Cos-7 Cells

pCMV-sports6-Smase may be transfected into mammalian Cos-7 cells. To transfect the cells with pCMV-sports6-Smase, a suitable number of cells, e.g., 4×10⁵ Cos-7 cells, are seeded in, e.g., a 25 cm² flask in a suitable amount of media, e.g., 4 ml. The media may be, e.g., Dulbecco's modified Eagle's medium with 10% heat inactivated fetal calf serum and 2 mM Glutamine. The cells are incubated until 90% confluent. Cells are then transfected with a suitable amount of pCMV-sports6-Smase, e.g., 5 microgram, and a suitable amount of lipofectamine 2000™, e.g., 16 microgram, in each flask followed by incubation for 48 h. Untransfected cells are exposed to lipofectamine and treated the same as above. At the end of the incubation, medium is collected and cells are lysed by a 50 mM Tris-HCl buffer containing 1 mM PMSF, 2 mM EDTA, 0.5 mM Dtt, 10 microgram/ml leupeptin, 10 microgram/ml aprotinine and 10 mM TC, or any other suitable buffer known in the art for the same purpose. Cells are normally kept on ice for, e.g., 10 minutes and then sonicated for 10 sec. After centrifugation at 12000 g for 10 minutes at 4° C., human Alk-Smase activity and protein concentrations were determined. One may use Cos-7 cells transfected with a mock-vector as a control. This may give transient expression of human Alk-Smase in Cos-7 cells for 48 h.

Expression of Alk-Smase in Yeast

The Alk-Smase cDNA may be amplified using vector pCMV-sports6-Smase as template as described above. Xho I and Not I sites may be introduced to the cDNA. The expression vector pPIC9 is commercially available from Invitrogen, Sweden. The Alk-Smase cDNA may be fused with the DNA fragment coding the a-factor signal peptide and regulated by a P_{AOX} promoter.

The pPIC9-SM plasmid may further be constructed by inserting the Alk-Smase cDNA digested by XhoI/Not I to pPIC9 digested by the same enzymes.

The recombinant yeast may then be obtained by transforming the pPIC9-SM to the pichia pastoris GS115 (Invitrogen AB) using electroporation and selected by His⁺ clones in His⁻ plate (example MD plate with 1.34% of YNB, 2% of Glucose, 1 μg/ml of biotin, 1.5% of agar) after culturing in 30° C. for 4-7 days.

The recombinant yeast may further be selected by culturing the transformant in BMGY (100 mM phosphate buffer, 2% yeast extract, 1% trypton and 1% Glycerol) medium at 30° C. for 24 hours.

Methanol may be added to 0.5% for 3 days to induce the expression of Alk-Smase and the produced Alk-Smase in the medium and the cells will be harvested. The Alk-Smase may be produced with the recombinant yeast through flask culture or fermentation, or any other suitable technique.

Expression of Recombinant Alk-Smase in E coli

In one embodiment recombinant Alk-Smase is expressed and prepared from E Coli.

The recombinant Alk-Smase may be produced as a glutathione-s-transferase fusion protein using in the art known fusion protein technique.

The Alk-Smase cDNA may be amplified by primer1

| | |
|---|---|
| primer1 | (5′ATGGATCCATGAGAGGCCCGGCCGTCCTCCT3′, |
| | Seq ID No 17) |
| and | |
| primer2 | (5′ACGTCGACTTACCAGCACCATAACAGCCAAG3′, |
| | Seq ID No 18) |

using vector pCMV-sports6-SMase as template.

A BamH I site and a Sal I site may be introduced to the cDNA. The pGEX-4T-1 containing glutathione-s-transferase gene may be used to construct expression vector, which is regulated by a P_{tac} promoter. The expression vector may be constructed by inserting the Alk-Smase cDNA digested by BamH I/Sal I. The expression vector may be transformed into E.coli BL21 by e.g. calcium chloride or Multioperator.

The transformed bacteria may then be plated in the ampicillin selective plate and the transformed E.coli, i.e., pGEX-4T-1-Smase, clones can be selected with restriction analysis and Alk-Smase activity assay after expanded in LB medium and induction by 5 mM IPTG (Isopropyl-d-Thiogalactoside). One E.coli pGEX-4T-1-Smase clone may be seeded in 25 ml LB medium with ampicillin and cultured overnight, and may be further inoculated in 500 ml LB. The cells may then be harvested and lysed.

The glutathione-s-transferase-Smase fusion protein is isolated by e.g. GSTrap™ FF affinity chromatography and cleaved by thrombin. The recombinant Alk-Smase is purified by GSTrap™ FF affinity chromatography and gel filtration chromatography.

Expression Vectors

Thus, the nucleotide sequence/sequences according to the invention may be present in a vector, such as an expression vector, which may be used for the production of human Alk-Smase, which has the capacity to hydrolyse Sm. The vector is typically derived from plasmid or viral DNA. A number of suitable expression vectors for expression in a host cells mentioned herein are commercially available or described in the literature. Any kind of vector may be used as long as it functions in a host cell which is capable of performing correct glycosylation and folding of human Alk-Smase. Examples are vectors enabling expression in E Coli, yeast or mammalian cells as described in the paragraphs above.

Other vectors for use in this invention include those that allow the nucleotide sequence encoding the polypeptide to be amplified in different copy numbers, such as high copy numbers or low copy numbers. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The vector may further comprise a DNA sequence enabling the vector to replicate in the host cell in question.

The vector may also comprise a selectable marker, e.g., a gene the product of which complements a toxin related deficiency in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g., ampicillin, kanamycin, tetracyclin, chloramphenicol, neomycin, hygromycin, zeocin or methotrexate.

The term “control sequences” is defined herein to include all components which are necessary or advantageous for the expression of the polypeptide of the invention. Each control sequence may be native or foreign to the nucleic acid sequence encoding the polypeptide. Such control sequences include, but are not limited to, a leader sequence, signal peptide, polyadenylation sequence, propeptide sequence, promoter (inducible or constitutive), enhancer or upstream activating sequence, signal peptide sequence, and transcription terminator. At a minimum, the control sequences include a promoter.

The presence or absence of a signal peptide will depend on the expression host cell used for the production of the polypeptide to be expressed (whether it is an intracellular or extra cellular polypeptide) and whether it is desirable to obtain secretion.

A Composition Comprising Human Alk-Smase

The present invention further comprises a composition comprising a human protein according to the invention capable of hydrolysing sphingomyelin, e.g., human Alk-Smase, or a nucleic acid according to the invention, or a human isolated or recombinant Alk-Smase according to the invention, and a biocompatible carrier or additive.

The human Alk-Smase may be isolated human Alk-Smase or recombinant Alk-Smase according to the invention.

In a further embodiment, the composition further comprises a buffer system ensuring an alkali pH of about 7.5-9.5, such as 7.5, 8.0, 8.5, 9.0, or 9.5.

In still a further embodiment, the protein according to the invention is a modified protein according to the suggested modifications above. Still, after such modifications, the enzyme remains is specific hydrolysing activities at the same, or higher, activity.

In still a further embodiment, the protein according to the invention is a part of Seq ID No 1, such as any part including the active site, i.e., Seq ID No 3. The part of the enzyme may in still further embodiments also be a modified protein having the same or increased activity as compared to the enzymes normal biological activity at defined enzymatic conditions, i.e., at a defined pH, and using a defined substrate.

In further embodiments the invention comprises the human Alk-Smase, in isolated or recombinant form, or parts or modifications thereof, with or without B-cer or lactase-phlorizin hydrolase as well as substrates for these enzymes. Such substrates are known in the art.

Furthermore, the present invention further comprises uses of a protein according to the invention, or a nucleic acid according to the invention, or a human isolated or recombinant Alk-Smase according to the invention, for the preparation of a pharmaceutical composition for the treatment of Smase related deficiencies/diseases such as celiac disease where the Alk-Smase activity is low due to the villous atrophy, in ulcerative colitis where the cancer risk is increased during long term follow up and in colon cancer, in the irritable bowel syndrome, and in patients running an increased risk of hereditary forms of colon cancers. Also included in the invention is the treatment of preterm infants vulnerable to necrotizing enteritis increasing the risk being counteracted by human milk since sphingomyelin is a major phospholipid in milk. Furthermore, treatment of cancers in the breast, prostate, lungs, skin, liver, stomach, thyroid gland, small bowel, pancreas and malignant tumours in lymphoid tissues, the musculo-skeletal system and brain are also included.

In a further embodiment, the pharmaceutical composition comprises a pharmaceutical acceptable carrier or additive. The pharmaceutical preparation according to the invention may be together with a pharmaceutically acceptable carrier and/or additives, such as diluents, preservatives, solubilizers, emulsifiers, and adjuvants useful in the pharmaceutical preparation disclosed in the present invention. Such pharmaceutically acceptable carrier and/or additives are known to the skilled man in the art.

Further, as used herein “pharmaceutically acceptable carriers” are well known to those skilled in the art and may include, but are not limited to, 0.01-0.05M phosphate buffer or 0.8% saline. Further, such pharmaceutically acceptable carriers may be aqueous or non-aqueous solutions, suspensions, and emulsions. Examples of non-aqueous solvents are propylene glycol, polyethylene glycol, vegetable oils such as olive oil, and organic esters such as ethyl oleate. Aqueous carriers include water, alcoholic/aqueous solutions, emulsions or suspensions, including saline and buffered media.

Preservatives and other additives may also be present, such as, for example, antimicrobials, antioxidants, chelating agents, inert gases and the like, as well as, but not limited to, other additives mentioned in the paragraphs below.

The composition, or pharmaceutical composition, according to the invention may, of course, in different embodiments contain relevant additives, such as electrolytes, fatty acids and amino acids. Other relevant additives are excipients, which are acceptable and compatible with the active ingredients, i.e., the protein human Alk-Smase according to the invention or parts thereof. Suitable excipients are, for example, water, saline, dextrose, sucrose, glycerol, ethanol, or the like and combinations thereof. In addition, if desired, the composition can contain minor amounts of auxiliary substances such as wetting or emulsifying agents, pH, buffering agents, which may enhance the effectiveness of the active ingredient.

In even further embodiments, the composition may include other relevant additives, such as filings and various buffers (e.g., Tris-HCI., acetate, phosphate) to set a fixed pH and ionic strength, and/or additives such as albumin or gelatine to prevent absorption to surfaces, detergents (e.g., Tween 20, Tween80, Pluronic F68, bile acid salts), solubilizing agents (e.g., glycerol, polyethyleneglycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol, sucrose).

Even further embodiments include covalent attachment of polymers such as polyethylene glycol to the composition, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellarvesicles, erythrocyte ghosts, or spheroplasts.

Further embodiments may be in suspensions or solutions. Also, the formats may be in capsules or tablets, such as chewable or soluble, e.g. effervescent tablets, as well as a powder, e.g., water soluble powder, flakes, granules or other dry formats known to the skilled man in the art, such as pellets, e.g. as micropellets, grains and granula.

Further embodiments include the composition or pharmaceutical composition in emulsified form. The active therapeutic ingredient may then be mixed with excipients, which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH, buffering agents, which may enhance the effectiveness of the active ingredient.

Treatment, prevention or alleviation using the composition according to the invention may be of any disease or disorder related to deficiencies in hydrolysing Sphingomyelin or defects of Alk-Smase. Examples are colon cancer and patients running an increased risk of hereditary forms of colon cancers, cancers in the breast, prostate, lungs, skin, liver, stomach, thyroid gland, small bowel, pancreas and malignant tumours in lymphoid tissues, the musculo-skeletal system and brain. Other diseases are the irritable bowel syndrome, Crohns disease, ulcerative colitis, collagenous colitis and lymphocytic colitis. Also, preterm infants are vulnerable to necrotizing enteritis have a risk being counteracted by human milk. Sphingomyelin is a major phospholipid in milk.

Administration Targets

The composition or pharmaceutical composition may be a composition for medical or veterinary use. As such, the administration targets may be a mammal, such as a mouse or a rat or any other rodent, a pig, a cat, a dog, a primate or half-ape such as the cotton tail tamarind. Also, the administration target may be a human being in need thereof.

Administration Doses and Routes

In the methods and use for manufacture of compositions of the invention, a therapeutically effective amount of the active component is provided. A therapeutically effective amount can be determined by the ordinary skilled medical or veterinary worker based on patient characteristics such as age, weight, sex, condition, complications, other diseases, etc., as is well known in the art.

The amount and dosages in pharmaceutical compositions may be from about 0.1 microgram to about 1 mg of human Alk-Smase protein capable of hydrolysing Sphingomyelin.

Administration may be performed in different ways depending on what species of vertebrate is treated, the condition of the vertebrate in need of said methods, and the specific indication to be treated.

Administration may be performed in a pharmaceutically acceptable dosage form. The composition or pharmaceutical composition may be administered intravenously as a bolus, or by continuous infusion over a period of time, by intramuscular, subcutaneous, intraperitoneal, oral, rectal, topical or inhalation routes.

The pharmaceutical compositions of this invention can also be administered as part of a combinatorial therapy with other agents.

### EXPERIMENTS

### Experiment 1

### Inhibition of Proliferation of HT 29 Cells Using Alk-Smase

Objective

The objective of the present example is to inhibit proliferation of a colon cancer cell line using human Alk-Smase.

Materials and Methods

Alk-Smase purified as described in the present invention.

Experimental Setup

Experimental conditions for the antiproliferative inhibition were that HT 29 cells were incubated with rat Alk-Smase at different doses for 18 h. The cell proliferation rates were measured by WST reagent.

HT29 human colon cancer cells were incubated in RMPI-1640 medium with L-glutamine containing antibiotics and 10% (v/v) heat inactivated fetal calf serum. At the exponential growth phase, the cells were incubated with purified rat Alk-Smase at different doses for 18 h. The cell proliferation rates were measured by WST reagent. Results are Mean±SD of three individual duplicate experiments. The figure shows that Alk-Smase dose-dependently inhibited cell growth.

Results and Discussion

FIG. 10 shows the effect of rat Alk-Smase on proliferation of colon cancer cells. Results are shown in FIG. 10 as a mean±SD of three individual duplicate experiments Human and rat alkaline Smase inhibit proliferation of the human colon cancer cell line HT 29 in cellular experiments.

### Experiment 2

Expression of Human Alk-Smase in Mammalian Cos-7 Cells

Objective

The objective of the present example is to express human Alk-Smase in mammalian Cos-7 cells.

Materials and Methods

Alk-Smase purified as described in the present invention.

Vector used is pCMV-sports6-Smase disclosed in the present invention.

Expression of Recombinant Alk-Smase in Mammalian Cos-7 Cells

pCMV-sports6-Smase is transfected into mammalian Cos-7 cells. To transfect the cells with pCMV-sports6-Smase 4×10⁵ Cos-7 cells are seeded in a 25 cm² flask in 4 ml of Dulbecco's modified Eagle's medium with 10% heat inactivated fetal calf serum and 2 mM Glutamine.

The cells are incubated until 90% confluent.

Cells are then transfected with 5 microgram pCMV-sports6-Smase and 16 microgram lipofectamine 2000™ in each flask followed by incubation for 48 h.

Untransfected cells are exposed to lipofectamine and treated the same as above.

At the end of the incubation, medium is collected and cells are lysed by a 50 mM Tris-HCl buffer containing 1 mM PMSF, 2 mM EDTA, 0.5 mM Dtt, 10 microgram/ml leupeptin, 10 microgram/ml aprotinine and 10 mM TC.

Cells are kept on ice, e.g., for 10 minutes, and then sonicated for 10 sec.

After centrifugation at 12000 g for 10 minutes at 4° C., human Alk-Smase activity and protein concentrations were determined.

Cos-7 cells transfected with a mock-vector is used as a control.

### RESULTS

This gives transient expression of human Alk-Smase in Cos-7 cells for 48 h. Further, the activity of human Alk-Smase increased by 30-fold in the cell extract and in the medium by 5-fold.

While the invention has been described in relation to certain disclosed embodiments, the skilled person may foresee other embodiments, variations, or combinations which are not specifically mentioned but are nonetheless within the scope of the appended claims.

All references cited herein are hereby incorporated by reference in their entirety.

1. A protein comprising a sequence selected from the group consisting of SEQ ID NO:1, a variant of SEQ ID NO:1, SEQ ID NO:4, and a variant of SEQ ID NO:4, wherein the sequence is capable of hydrolysing sphingomyelin.

2. The protein according to claim 1, wherein the sequence is capable of hydrolysing sphingomyelin at pH 7.5-9.

3. The protein according to claim 1, wherein the sequence has less than 50% of its hydrolysing activity at pH less than 7.5.

4. The protein according to claim 1, wherein the variant of SEQ ID NO:1 has at least 80% identity with SEQ ID NO:1 and the variant of SEQ ID NO:4 has at least 80% identity with SEQ ID NO:4 SEQ ID NO: 4.

5. A nucleotide sequence encoding the protein according to claim 1.

6. The nucleotide sequence according to claim 5, wherein the nucleotide sequence comprises SEQ ID NO: 2 or SEQ ID NO: 5.

7. A recombinant expression and secretion vector, comprising:
a polynucleotide encoding a secretion signal peptide;
a DNA sequence which promotes transcription in a host cell located upstream from the polynucleotide encoding the secretion signal peptide;
a DNA sequence encoding a protein according to claim 1 in a translation reading frame with said polynucleotide encoding the secretion signal peptide; and
a transcription terminator sequence located downstream from the DNA sequence encoding said protein.

8. A host cell, comprising;
the recombinant expression system according to claim 7, wherein the host cell expresses Alk-Smase.

9. The host cell according to claim 8, wherein the host cell is selected from the group consisting of a bacteria, a mammalian cell and a yeast cell; and
in the absence of the recombinant expression system according to claim 7, the host cell does not normally produce an Alk-Smase.

10. A method for isolation of human Alk-Smase protein, the method comprising the steps of:
providing a small intestinal or colon content from a human;
homogenising the small intestinal or colon content;
purifying Alk-Smase from the homogenized content using DEAE Sephadex chromatography;
purifying the Alk-Smase using Uno anion exchange chromatography; and
purifying the Alk.Smase using hydrophobic chromatography, thereby isolating the human Alk-Smase protein.

11. A method for preparation of recombinant Alk-Smase protein capable of hydrolysing sphingomyelin, the method comprising the steps of:
providing a host cell according to claim 8 and a host cell growth medium;
preparing a host cell culture;
culturing the host cell culture; and
harvesting the host cell culture and recovering the human recombinant Alk-Smase.

12. The method according to claim 11, wherein the Alk-Smase protein is recovered from the culture medium or the host cells.

13. An isolated Alk-Smase protein, comprising the protein according to claim 1, wherein the protein has an active site with the amino acid sequence AFVTMTSPCHFTLVTGKY (SEQ ID NO: 3) or a variant thereof.

14. A composition, comprising: a protein according to claim 1; and a biocompatible carrier or additive.

15. A method for treating colon cancer, comprising:
administering a composition comprising at least one of a protein according to claim 4, a nucleic acid according to claim 5, and an isolated Alk-Smase according to claim 12 to a patient.

16. A kit, comprising:
the protein according to claim 1 or the isolated protein according to claim 13; and
a stabiliser.

17. The kit according to claim 16, wherein the protein is in a lyophilised form or freeze-dried form.

18. The method according to claim 12, wherein the Alk-Smase protein is recovered after separating the host cells from the culture medium.

19. A composition, comprising:
a nucleic acid according to claim 5; and
a biocompatible carrier or additive.

20. A composition, comprising:
an isolated Alk-Smase according to claim 12; and
a biocompatible carrier or additive.
